(19) United States
(12) Patent Application Publication
(10) Pub. No.: US 2019/0139440 A1
(43) Pub. Date: May 9, 2019

(54) Cancer Prevention/Improvement Advice Device

(71) Applicant: Ryozo SAITO, Tokyo (JP)

(72) Inventor: Ryozo SAITO, Tokyo (JP)

(21) Appl. No.: 16/077,038

(22) PCT Filed: Feb. 9, 2017
(86) PCT No.: PCT/JP2017/004701

§ 371 Date: Dec. 28, 2018

(30) Foreign Application Priority Data
Feb. 11, 2016 (JP) 2016-024257

(51) Int. Cl. G09B 19/00; G06F 16/23; G16H 10/20; G16H 20/30; G16H 20/60; A61B 5/11; A61B 5/00

(52) U.S. Cl. CPC G09B 19/00; G06F 16/2379; G16H 10/20; G16H 20/30; G16H 20/60; A61B 5/4866; A61B 5/4806; A61B 5/4872; A61B 5/486; G09B 19/0092; A61B 5/1118

(57) ABSTRACT

There have been no devices and systems available from which one can easily receive, without stress, advice on lifestyle guidelines for preventing cancer, preventing recurrence, and promoting improvement without relying on a medical specialist, etc., in daily life. In order to solve the above problem, a cancer prevention/improvement advice device is provided with: an advice selection unit for retaining an advice selection rule, which is a rule for selecting advice directed at the purposes of cancer prevention and improvement using information affecting the physiological activity of a user and/or history information of lifestyle factor information that indicates the degree of health, and selecting advice using the retained advice selection rule with the history information of the lifestyle factor information and a lifestyle factor information optimum value retained for the lifestyle factors being used as variables; and an advice output unit for outputting advice selected by the selection unit.

### RELATED APPLICATIONS

This application claims priority to International Patent Application No. PCT/JP2017/004701, International Filing Date Feb. 9, 2017, and claims benefit of and priority to JP Patent Application No. 2016-024257 filed Feb. 11, 2016 both of which are hereby incorporated herein by reference in their entireties.

### BACKGROUND

The present invention pertains to a cancer prevention/improvement advice device which outputs advice for more closely approaching optimum values of various lifestyle factors for the purpose of cancer prevention and improvement. Recently, it has become clear that many cancers are lifestyle-related diseases caused by preventable lifestyle habits and environmental factors, and that the risks for such cancers increase along with age. For example, on the “Cancer Information Service” website of the Center for Cancer Control and Information Services of the National Cancer Center, the newest and most correct information on cancer is presented for the general public (see non-patent document 1). Here, the National Cancer Center indicates, “From research thus far, we now know that many of the causes of cancer relate to daily lifestyle habits such as tobacco use, alcohol, and meals.” These lifestyle-related diseases are caused by lifestyle habits such as eating habits and exercise habits over many years, and therefore in order to achieve prevention and improvements, records must be kept for a long period of time, and the details must be examined. Furthermore, these records must be reported to doctors and other healthcare professionals, and advice and guidance must be received. However, in actuality, it is extremely cumbersome to maintain a record of daily eating habits and exercise, and the frequency of receiving guidance from healthcare professionals is around several times per year. In recent years, devices and systems that support improvements in lifestyle habits have been proposed. For example, patent document 1 describes that a user's biological data and lifestyle data are collected, and advice from a specialist can be promptly received, that data can be accumulated over a long period of time and analyzed, and that a patient's lifestyle habits and lifestyle deviations can be understood, and medical care services can be provided (see patent document 1).

Incidentally, recently, several medical cases have been reported in which there have been effects in so-called cancer improvements such as the progression of cancer being suppressed and cancer tumors becoming smaller through improvements in lifestyle habits. For example, non-patent document 2 describes that “when low risk prostate cancer patients engaged in exercise, meditation, and healthy eating habits (Mediterranean diet) for three months, of the genes relating to prostate cancer, 501 gene expressions were improved,” and discloses that cancer related gene expression is improved through improvements in lifestyle habits. This result suggests that cancer improvements can be targeted through improvements in lifestyle habits and are not limited to the medical care by a specialized doctor.

Moreover, non-patent document 3 describes, “When ketosis (a condition in which ketone bodies in the blood have increased at a physiological range due to a carbohydrate-restricted diet) was attempted under a sugar-restricted diet in which carbohydrates were restricted to 5% of the overall calories of meals in order to suppress insulin with respect to metastatic cancer of ten patients, one of the ten patients with metastatic cancer went into remission, and another three experienced a suppression of disease progression. On the other hand, cancer progressed in medical cases for which insulin could not be suppressed,” and disclosed that metastasis risk is reduced through ketosis. Accordingly, the results of non-patent document 3 also suggest that the risk of metastasis can be reduced through meal guidance for restricting the amount of carbohydrates that are consumed. On the other hand, the excessive secretion of ketone bodies in the urine can be confirmed through a qualitative analysis or quantitative measurement of ketone bodies in the urine, and it is known that ketosis can be evaluated by combining a controlled diet in which carbohydrate consumption is restricted with an analysis of ketone bodies in the urine. This suggests that cancer improvements can be targeted if meal information and biological data can be understood and advice on improvement measures can be received without directly receiving guidance from a doctor who is a specialist.

In this manner, guidance for improving lifestyle habits is beginning to attract attention even with respect to medical treatments for cancer. It was discovered 50 years ago that cancer lives on sugar content (Warburg effect), and it has been shown that sugar is a form of energy that is preferentially used by cancer cells, and that by restricting carbohydrates, often times cancer growth directly decreases. It is also known that an effect of suppressing cancer occurs by minimizing the amount of sugar and insulin in the blood through carbohydrate restriction. Furthermore, as examples in which the progression of cancer was suppressed by improvements in eating habits, cases of limiting salt content and increasing vegetable consumption, of limiting proteins and increasing vegetable consumption, and of limiting milk products have also been presented.

Research is also advancing on cancer and inflammation, and it is known that inflammation plays a role in some metastasis of cancer. Obesity and arteriosclerosis are also considered to be the main causes of inflammation, and it is thought that preventing obesity and arteriosclerosis by improving lifestyle habits can lead to cancer prevention and improvements. When considered based on eating habits, omega-3 fatty acids including docosahexaenoic acid, eicosapentaenoic acid, and gamma-linolenic acid are said to have an anti-inflammatory effect, but because it is difficult to ingest these in ordinary meals, the matter of proactively ingesting food products containing omega-3 fatty acids is being advocated. Omega-3 fatty acids have been found to have an effect of delaying and stopping the growth of certain cancer metastatic cells, and an extension of the lifespan of cancer patients and an improvement in the quality of life (QOL) is anticipated.

Based on this type of knowledge, in order to achieve cancer prevention and improvements, it is now recommended that, for example, with regard to food and drinks, that diets be centered on plant-based food products and unprocessed foods, that the intake of milk products be minimized, that diets be low in carbohydrates and low in salt, and that alcohol consumption be reduced or abstained. Furthermore, it is noted that engaging in an appropriate amount of exercise, and reducing stress to maintain a stable mental state are also helpful for cancer prevention and improvements. However, while even medical specialists are beginning to focus attention on improving lifestyle habits, currently, medical specialists are swamped with diagnosis and medical treatments, and are not providing guidance for improving lifestyle habits to patients and their families.

### PRIOR ART DOCUMENTS

### Patent Documents

Patent Document 1: WO 2003/103485

### Non-Patent Literature

Non-Patent Literature 1: Center for Cancer Control and Information Services of the National Cancer Center, “Cancer Information Service” Internet <Homepage: see Preventive Health Screening section, Cancer Prevention Methods for Japanese People>
Non-Patent Literature 2: “Changes in prostate gene expression in men undergoing an intensive nutrition and lifestyle intervention”, Ornish et al., PNAS, Jun. 17, 2008, vol. 105, no. 24, pp. 8369-8374
Non-Patent Literature 3: Fine E J, Segal-Isaacson C J, Feinman R D, Herszkopf S, Romano M C, Tomuta N, Bontempo A F, Negassa A, Sparano J A, Nutrition, 2012 October; 28(10):1028-35, doi: 10.1016/j.nut.2012.05.001, Epub, 26 Jul. 2012.

### SUMMARY OF INVENTION

### Problem to be Solved by the Invention

Improvements in lifestyle habits are expected to exhibit an effect not only on preventing cancer prevention, but also improving cancer conditions, but lifestyle habits are not easily improved. It is important that lifestyle habits be inspected to check for the presence of any deviations in everyday meals, exercise, and sleep patterns and for the presence or lack of stress, that changes in biological data such as body weight and blood pressure be verified, and that improvements be made and continued a little at a time. Therefore, it is important that conditions such as meals, exercise, sleep, stress, and biological data be examined, and that advice for improvements be received, as appropriate. However, conventional devices and systems like that described by patent document 1 are merely for the purpose of making it easier to receive advice from specialists, and do not realize a function of issuing advice for the purpose of preventing and improving cancer.

Therefore, a problem to be solved by the present invention is to provide a device that is capable of selecting and outputting advice for bringing various lifestyle factors such as meals and exercise closer to optimum values for the purpose of cancer prevention and improvement through the present invention, and to provide a cancer prevention/improvement advice device that enables a user to receive advice from a smartphone or other electronic device that is ordinarily close to the user.

### Means for Solving the Problem

In order to solve the abovementioned problem, of the present invention, a first aspect provides a cancer prevention/improvement advice device including: a lifestyle factor information acquisition unit for acquiring, from a user, lifestyle factor information, which is information affecting the physiological activity of the user and/or information indicating a degree of health of the user; a lifestyle factor information accumulation unit for accumulating the acquired lifestyle factor information as history information; a lifestyle factor information optimum value retaining unit for retaining lifestyle factor information optimum values, which are optimum values for each lifestyle factor directed at the purpose of cancer prevention and improvement; an advice retaining unit for retaining a plurality of advice items for more closely approaching the optimum value of each lifestyle factor information; an advice selection rule retaining unit for retaining advice selection rules, which are rules for selecting the retained advice using the history information of the lifestyle factor information and the lifestyle factor information optimum values retained for the lifestyle factors as variables; an advice selection unit for selecting advice using the retained advice selection rules with the history information of the lifestyle factor information and the lifestyle factor information optimum values retained for the lifestyle factors being used as variables; and an advice output unit for outputting advice selected by the selection unit.

In addition, a second aspect provides the cancer prevention/improvement advice device according to claim 1, wherein the lifestyle factor information acquisition unit has a meal information acquisition means for acquiring meal information, which is a type of lifestyle factor information, from a photograph of a meal.

Moreover, a third aspect provides the cancer prevention/improvement advice device according to claim 1 or 2, wherein the lifestyle factor information acquisition unit has an exercise data acquisition means for acquiring exercise data, which is a type of lifestyle factor information, from an exercise amount measuring instrument such as a pedometer.

Moreover, a fourth aspect provides the cancer prevention/improvement advice device according to any one of claims 1 to 3, wherein the lifestyle factor information acquisition unit has a sleep data acquisition means for acquiring sleep data, which is a type of lifestyle factor information, from a sleep measuring instrument.

Moreover, a fifth aspect provides the cancer prevention/improvement advice device according to any one of claims 1 to 4, wherein the lifestyle factor information acquisition unit has a stress data acquisition means for acquiring stress data, which is a type of lifestyle factor information, from a stress measuring instrument.

Moreover, a sixth aspect provides the cancer prevention/improvement advice device according to any one of claims 1 to 5, wherein the lifestyle factor information acquisition unit has a biological data acquisition means for acquiring biological data, which is a type of lifestyle factor information, from a body composition meter.

Furthermore, a seventh aspect provides an operation program for a prevention/improvement advice device that is a cancer computer, the operation program including: a lifestyle factor information acquisition step for acquiring lifestyle factor information, which is information affecting the physiological activity of a user and/or information indicating the degree of health of a user, from the user; a lifestyle factor information accumulation step for accumulating the acquired lifestyle factor information as history information; a lifestyle factor information optimum value retaining step for retaining lifestyle factor information optimum values, which are optimum values for each lifestyle factor directed at the purpose of cancer prevention and improvement; an advice selection step for selecting advice from a plurality of advice for more closely approaching the retained optimum value of each lifestyle factor information, using advice selection rules, which are rules for selecting, as variables, the history information of the lifestyle factor information and the lifestyle factor information optimum values retained for the lifestyle factors; and an advice output step for outputting advice selected by the selection unit.

The following system is conceivable as a system for increasing advice precision through big data processing.

Namely, as an eighth aspect, the cancer prevention/improvement advice device according to claims 1 to 6 is proposed, wherein for a case in which the lifestyle factor information is acquired as a numeric value, the lifestyle factor information accumulation unit includes a first statistical processing means for determining, on the basis of past numeric values, an average value, a dispersion, a deviation value of data each time, a t-distribution, an F-distribution, and a chi-squared distribution.

Furthermore, as a ninth aspect, the cancer prevention/improvement advice device based on the eighth aspect is proposed, further having, for a case in which the lifestyle factor information is acquired as a numeric value, a lifestyle factor information cross-correlation coefficient acquisition unit for acquiring a lifestyle factor information cross-correlation coefficient, which is a cross-correlation coefficient for the lifestyle factor information accumulated by the lifestyle factor information accumulation unit.

Furthermore, as a tenth aspect, the cancer prevention/improvement advice device based on the eighth or ninth aspect is proposed, wherein the advice selection rule retaining unit has an advice first selection rule changing means for changing the retained advice selection rule on the basis of an average value, a dispersion, a deviation value of data each time, a t-distribution, an F-distribution, and a chi-squared distribution determined by the first statistical processing means of the lifestyle factor information accumulation unit.

Furthermore, as an eleventh aspect, the cancer prevention/improvement advice device according to the eighth or ninth aspect is proposed, wherein the lifestyle factor information accumulation unit further includes: an advice associated lifestyle factor information accumulation means for accumulating lifestyle factor information that is associated with advice output from the advice output unit and acquired; and an advice lifestyle factor information correlation coefficient acquisition unit for acquiring an advice lifestyle factor information correlation coefficient, which is a correlation coefficient between the associated advice and the lifestyle factor information.

Furthermore, as a twelfth aspect, the cancer prevention/improvement advice device according to the eleventh aspect is proposed, wherein the advice selection rule retaining unit includes a second advice selection rule changing means for changing the retained advice selection rule on the basis of the advice lifestyle factor information correlation coefficient acquired by the advice lifestyle factor information correlation coefficient acquisition unit.

As a thirteenth aspect, a cancer prevention/improvement advice system is proposed, the system including: the cancer prevention/improvement advice device according to any one of the first to sixth and the eighth to twelfth aspects further having: a history information output unit for outputting, through a network, the history information of the lifestyle factor information accumulated by the lifestyle factor information accumulation unit; a history information acquisition unit for acquiring, through a network, the history information output from the history information output unit; and a central server having: a central server correlation coefficient calculation unit for statistically processing the acquired history information, and calculating a lifestyle factor information cross-correlation coefficient, and a central server correlation coefficient output unit for outputting the calculated correlation coefficient.

Furthermore, as a fourteenth aspect, the cancer prevention/improvement advice system according to the thirteenth aspect is proposed, wherein the central server includes a second statistical processing unit for determining, on the basis of the acquired history information, an average value of each lifestyle factor information, a dispersion, a deviation value of data each time, a t-distribution, an F-distribution, and a chi-squared distribution.

Furthermore, as a fifteenth aspect, the cancer prevention/improvement advice system according to the thirteenth or fourteenth aspect is proposed, wherein the central server further includes: an advice selection command acquisition unit for acquiring an advice selection rule change command, which is a command for changing the advice selection rule retained by the advice selection rule retaining unit for each cancer prevention/improvement advice device on the basis of the correlation coefficient calculated by the central server correlation coefficient calculation unit; and an advice selection rule change command output unit for outputting the acquired advice selection rule change command to each cancer prevention/improvement advice device; and each cancer prevention/improvement advice device further includes: an advice selection rule change command acquisition unit for acquiring an advice selection rule change command output from an advice selection rule change command output unit; and an advice selection rule change unit for changing, on the basis of an acquired advice selection rule change command, an advice selection rule retained by the advice selection rule retaining unit.

Furthermore, measures to using artificial intelligence (AI) to learn advice rules, increase the precision of advice selection and make updates are conceivable.

Namely, as a sixteenth aspect, a cancer prevention/improvement advice device is provided, the device including: a lifestyle factor information acquisition unit for acquiring, from a user, lifestyle factor information, which is information affecting the physiological activity of the user and/or information indicating the degree of health of the user; a lifestyle factor information accumulation unit for associating the acquired lifestyle factor information with a below-described advice information that is output corresponding to the acquisition period of the lifestyle factor information, and accumulating the associated lifestyle factor information as history information; a lifestyle factor information evaluation unit for evaluating the lifestyle factor information using a below-described lifestyle factor information optimum value, and obtaining evaluation results; an advice results learning unit for learning a relationship between the evaluation results and the advice information corresponding to the evaluated lifestyle factor information; a lifestyle factor information optimum value retaining unit for retaining lifestyle factor information optimum values, which are optimum values for each lifestyle factor directed at the purpose of cancer prevention and improvement; an advice retaining unit for retaining a plurality of advice for more closely approaching the optimum value of each lifestyle factor information; an advice selection rule retaining unit for retaining advice selection rules, which are rules for selecting the retained advice using the history information of the lifestyle factor information, the lifestyle factor information optimum values retained for the lifestyle factors, and the learning results; an advice selection unit for selecting advice using the retained advice selection rules with the history information of the lifestyle factor information and the lifestyle factor information optimum values retained for the lifestyle factors being used as variables; and an advice output unit for outputting advice selected by the selection unit.

Furthermore, as a seventeenth aspect, the cancer prevention/improvement advice device according to claim 2 is proposed, further including a new advice recording unit for recording new advice in the advice retaining unit.

These lifestyle factor information items may include, for example, any one or more types of information including, as information on body diagnosis results, body measurement results (BMI, abdominal girth, visceral fat, body fat percentage), body status examination results (blood pressure, visual acuity, ocular pressure, pulmonary function, hearing capability, hair conditions), urinalysis results, stool examination results, number of times of stool passage, stool color, stool characteristics, blood biochemical test results, serological examination results, tumor marker examination results, diabetes mellitus examination results, biopsy examination results, fundus camera examination results, breast examination results, ultrasound examination results, gastrointestinal tract examination results, chest X-ray examination results, CT examination results, MRI examination results, PET examination results, rectal examination results, rectal temperature, electrocardiogram examination results, equilibrium function examination results, arteriosclerosis evaluation results, osteoporosis examination results, female hormone examination results, risk marker examination results, and genetic information. Moreover, daily life information may include any one or more types of information including the light quality and brightness of the living environment, amount of noise in the living environment, room temperature of the living environment, humidity of the living environment, light quality and brightness of the workplace environment, amount of noise in the workplace environment, room temperature of the workplace environment, humidity of the workplace environment, meal content, calorie intake from meals, food product ingredients and amount ingested with meals, number of meals, timing of meals, details of ingested supplements, intake calories from ingested supplements, ingested supplement ingredients and amounts from ingested supplements, number of times of ingestion of ingested supplements, ingestion timing of ingested supplements, quality of sleep, amount of sleep, type of exercise and amount of exercise, quality of work and amount of work, working hours, amount of movement at work, commute quality and amount, evaluation results from company at work (salary increase, job promotion, job transfer, change in assignment), presence or lack of unemployment and career change, quality and amount of training to maintain and increase physical strength, quality and amount of walking around town, quality and amount of shopping, quality and amount of contact with the natural environment, quality and amount of entertainment, quality and amount of friendships, quality and amount of marital relations, type and amount of accidents, presence or lack of residence changes, presence or lack of marriage and divorce, purchase history of expensive articles and expensive goods, quality and amount of learning for obtaining qualifications, presence or lack of qualification acquisition, and change in relationship with customers for cases of company management and self-employment. Illness and injury information may include any one or more types of information including the types and amounts of injuries and illnesses, types and amounts of drugs that are being taken in accordance with injuries and illnesses, frequency and time length of hospital visits according to injuries and illnesses, types and amounts of diagnoses performed in accordance with injuries and illnesses, types and amounts of surgeries performed in accordance with injuries and illnesses, devices carried on the body in accordance with injuries and illnesses, assistance tools, type and amount of bandages and the like. In addition, mental status information may include any one or more types of information including the voice quality of the user, the voice magnitude of the user, the amount of speech by the user, the status of autonomic nerves, the mood of the user, the level of satisfaction with sex life, the embraced image of an item, mood when getting out of bed, mood when falling asleep, mood during sleep, mood during work, mood on holidays, mood during meals, mood when defecating, the effective body temperature, the coldness of hands and feet, and the ease of respiration.

### Effect of the Invention

According to the present invention, a cancer prevention/improvement advice device capable of acquiring lifestyle factor information from a user and outputting advice for more closely approaching optimum values of various lifestyle factors for the purpose of cancer prevention and improvements can be provided.

### BRIEF DESCRIPTION OF THE DRAWINGS

FIG. 1 is an image illustrating an example of a functional block of a cancer prevention/improvement advice device of the present invention.

FIG. 2 is a schematic view showing an example of the hardware architecture of the cancer prevention/improvement advice device of the present invention.

FIG. 3 is a flowchart showing the processing flow of the cancer prevention/improvement advice device of the present invention.

FIG. 4 is an image showing an example for acquiring meal information, which is type of lifestyle factor information, with respect to an Embodiment 1.

FIG. 5 is a flow chart showing an example of the processing flow for acquiring lifestyle factor information according to Embodiment 1.

FIG. 6 is an image showing an example of an ingredient table for curry rice according to Embodiment 1.

FIG. 7 is an image showing an example for acquiring exercise data, which is a type of lifestyle factor information, according to an Embodiment 2.

FIG. 8 is an image showing an example for acquiring sleep data, which is a type of lifestyle factor information, according to an Embodiment 3.

FIG. 9 is an image showing an example of lifestyle factor information displayed on a screen of a smartphone according to the Embodiment 3.

FIG. 10 is an image showing an example in which stress data, which is a type of lifestyle factor information, is output with respect to an Embodiment 4.

FIG. 11 is a schematic view of an eye for grasping pupillary changes with respect to Embodiment 4.

FIG. 12 is a flowchart showing an example of the processing flow for acquiring lifestyle factor information according to Embodiment 4.

FIG. 13 is an image showing an example for acquiring biological data, which is a type of lifestyle factor information, with respect to an Embodiment 5.

FIG. 14 is an image showing another example of a functional block for the cancer prevention/improvement advice device of the present invention.

FIG. 15 is an image for explaining the mechanism for acquiring advice with respect to an Embodiment 6.

FIG. 16 is an image showing the functional configuration of an Embodiment 7.

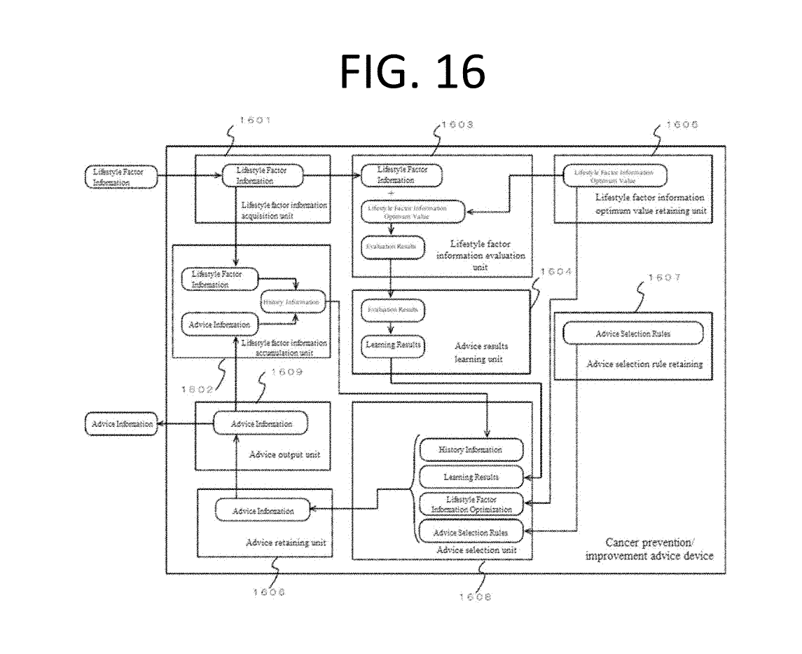

FIG. 17 is an image showing specific processing for selecting advice according to Embodiment 7.

FIG. 18 is an image showing an example of genetic information.

FIG. 19A is a first image showing lifestyle factor information optimum values.

FIG. 19B is a second image showing lifestyle factor information optimum values.

FIG. 19C is a third image showing lifestyle factor information optimum values.

FIG. 19D is a fourth image showing lifestyle factor information optimum values.

FIG. 20 is an image showing the hardware architecture of Embodiment 7.

FIG. 21 is a flowchart showing the processing flow of Embodiment 7.

### REFERENCE NUMERALS

0100: cancer prevention/improvement advice device
0101: lifestyle factor information acquisition unit
0102: lifestyle factor information accumulation unit
0103: lifestyle factor information optimum value retaining unit
0104: advice retaining unit
0105: advice selection rule retaining unit
0106: advice selection unit
0107: advice output unit

### MODE FOR CARRYING OUT THE INVENTION

### <Overview>

In the present invention, the “cancer prevention/improvement advice device of the present invention” is an invention which gathers information from daily life without impacting physiological activity, outputs advice on a daily basis for preventing the onset of cancer as well as cancer recurrence, and decreasing and eliminating cancer even in cases for which onset has occurred, and thereby facilitates the health maintenance of a user and prevents cancer onset and recurrence.

### <Configuration>

The “cancer prevention/improvement advice device” of the present invention (hereinafter, abbreviated as the present device) is provided with a lifestyle factor information acquisition unit, a lifestyle factor information accumulation unit, a lifestyle factor information optimum value retaining unit, an advice retaining unit, an advice selection rule retaining unit, an advice selection unit, and an advice output unit.

Note that a functional block of the present device and system described hereinafter can be realized as a combination of hardware and software. More specifically, if a computer is used, examples of the hardware and software include a CPU and main memory, a bus, or a secondary storage device (hard disk, nonvolatile memory, CD, DVD, or other storage media and a drive for reading these media, etc.), an input device used for inputting information, a printing device and a display device, other external peripheral devices, and other such hardware architectural parts, and interfaces for the external peripheral devices, communication interfaces, a drive program for controlling these hardware items, other application programs, applications for user interfaces, and the like. Furthermore, through computation processing by the CPU in accordance with a program expanded in the main memory, data input from an input device or other interface, etc. and retained in memory or on a hard disk is processed and accumulated, and commands for controlling each piece of hardware and the software are generated. Alternatively, the functional block of the system may be realized through dedicated hardware.

Each embodiment described in the present specification can be realized not only as a device, but also as a method. In addition, a portion of this type of device can be configured as software. The software product used for executing this type of software on a computer, and the recording medium on which this product is fixed are also of course included within the technical scope of each embodiment described in the present specification (the same applies throughout the entirety of the present specification).

The embodiments of the present invention are devices having the abovementioned configuration, and can be realized through a smartphone, wearable terminal, tablet terminal, PC terminal and other various terminal devices and servers, etc. on a network, but the present invention is not limited to these embodiments in any way, and can be implemented with various aspect within a scope that does not depart from the gist of the present invention. Smartphones, wearable terminals, tablet terminals, PC terminals, and other various types of terminal devices are typically present as devices that process information using a built-in computer. In addition to these typical devices, the various types of terminals also include cell phones, gaming devices, navigation devices, and the like. Furthermore, the term “smartphone” refers to a high performance cell phone that uses a touch panel as a main user interface device.

### <Description of the Configuration>

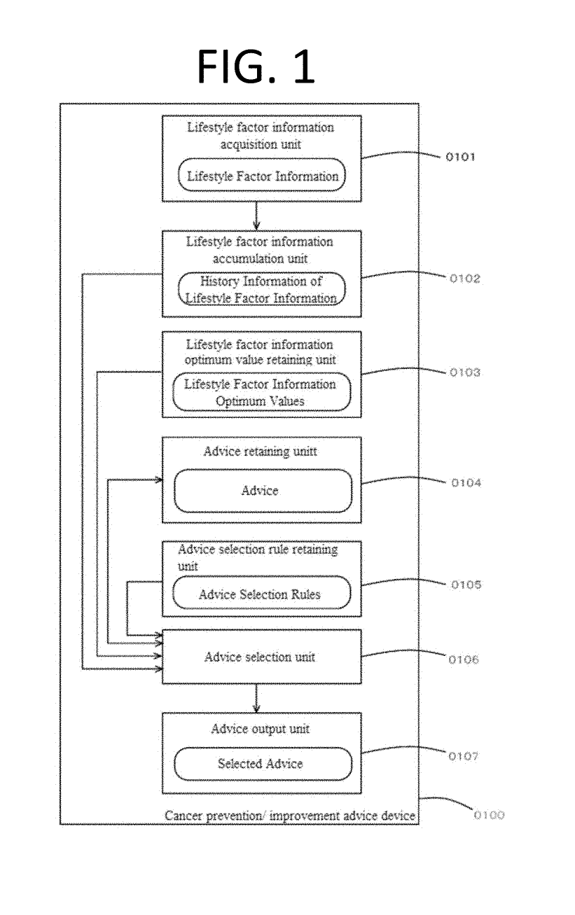

FIG. 1 is an image illustrating an example of a functional block of a cancer prevention/improvement advice device of the present invention. Each configurational element is described while referencing this image. As shown in FIG. 1, first, a lifestyle factor information acquisition unit (0101) directly or indirectly acquires from a user or external device, lifestyle factor information, which is information affecting the physiological activity of the user and/or information indicating a degree of health of the user. The acquired lifestyle factor information is sent to a lifestyle factor information accumulation unit (0102), which accumulates the information as history information. In addition, lifestyle factor information optimum values, which are optimum values for each lifestyle factor directed at the purpose of cancer prevention and improvement, and which correspond to these lifestyle factor information items or lifestyle factor information items that are synthesized internally, are retained in a lifestyle factor information optimum value retaining unit (0103). In addition, an advice retaining unit (0104) for retaining a plurality of advice for more closely approaching the optimum value of each lifestyle factor information is provided, and advice selection rules, which are rules for selecting the retained advice using the history information of the acquired lifestyle factor information and the lifestyle factor information optimum values retained for the lifestyle factors as variables, are retained in an advice selection rule retaining unit (0105). Furthermore, advice selection rules that are rules for selecting advice for which the history information of the acquired lifestyle factor information is retained as a variable may also be retained by the advice selection rule retaining unit. An advice selection unit (0106) for selecting advice using the retained advice selection rules with the history information of the lifestyle factor information and the lifestyle factor information optimum values retained for the lifestyle factors being used as variables is also provided, and advice selected by the selection unit is output from the advice output unit (0107). The cancer prevention/improvement advice device according to the present invention is configured as described above.

The “lifestyle factor information acquisition unit” has a function of acquiring, from a user, lifestyle factor information, which is information affecting the physiological activity of the user and/or information indicating the degree of health of the user. Ordinarily, this function is realized using a camera function, a communication function, a video recording function, a sound accumulation function, a motion sensor function, a temperature measurement function, an illuminance measurement function, a humidity measurement function, and the like. The acquired lifestyle factor information is sent to the lifestyle factor information accumulation unit and accumulated. The accumulation is carried out in association with the date and user. In addition, for cases in which the present device is configured with a server on a network, the lifestyle factor information acquisition unit becomes an interface on the server for acquiring information from the camera function, video recording function, sound accumulation function, motion sensor function, temperature measurement function, illuminance measurement function, and humidity measurement function, etc.

Here, “lifestyle factor information” refers to information with the potential to affect the physiological activity of a user, and/or information that can be used to measure the degree of health of the user. Therefore, lifestyle factor information includes both information that becomes a cause of the degree of health and information indicating the degree of health as a result obtained by the cause thereof. In the present invention, lifestyle factor information includes, for example, information such as meal information, exercise data, sleep data, stress data, and biological data. Note that lifestyle factor information may also include information such as radiation doses, radiation strength, ultraviolet light doses, ultraviolet light strength, and electromagnetic wave strength. These may be acquired from the lifestyle factor information acquisition unit via a network, and devices such as a radiation sensor, ultraviolet light sensor, and electromagnetic wave sensor may be provided as lifestyle factor information acquisition units for the present invention.

“Meal information” refers, for example to information such as the time that eating and drinking were started, the time that eating and drinking were ended, the types and amounts of food and beverages that were consumed, the number of calories that were consumed, and the types and amounts of food ingredients in a food product. Examples of information regarding types of food ingredients include carbohydrates, dietary fiber, minerals, vitamins, other proteins, fats, and the types and percentages of fatty acids contained in fats. Examples of minerals include sodium, potassium, calcium, magnesium, phosphorous, silicon, zinc, iron, copper, manganese, selenium, chromium, and sulfur, and examples of vitamins include vitamins such as the vitamin B group, vitamin D, vitamin A, and vitamin K. Meal information also includes, for example, the consumption time and amounts of various substances such as caffeine, alcohol (Japanese sake, shochu, beer, whiskey, wine, brandy, etc.), tobacco, cigars, and various types of drugs (including chemotherapy drugs). Meal information also includes harmful substances such as, for example, harmful heavy metals, trans-fatty acids, PCB, environmental hormones, and agrochemicals.

These meal information details can be computed by using image recognition technology to analyze photos captured using a camera function of a smartphone, tablet terminal, or other such lifestyle factor information acquisition unit. For example, if a photograph is of spaghetti, the product ingredients and amounts that are standardly contained in spaghetti become meal information, which is a type of lifestyle factor information. In addition, rather than using photographs, an applicable meal can be selected from a pull-down menu or other such lifestyle factor information acquisition unit to thereby acquire the meal information. Alternatively, a meal menu can be input verbally using a voice recognition function and a microphone, which is a lifestyle factor information acquisition unit, to thereby acquire the meal information.

Furthermore, in the case of industrially mass produced food products (instant ramen, udon, soba, spaghetti and support materials of these, candy, canned beer, sushi, home delivered sushi, chilled Japanese box lunch, Japanese box lunch and rice balls, salads and boiled food, deep-fried food, grilled chicken, snacks and side dishes, bread loaves, bread rolls and breadsticks, sweetened buns, bread as a side dish, donuts, baked sweets and western confectioneries, chilled light meals, pizza and pizza sauce, jams and honey, toppings, frozen foods, ice-cream, desserts, sweets, milk products, fermented soybeans, tofu, konnyaku, Japanese picked vegetables, kimchi, processed fish meat, chilled food products, dressings, soups, retort food products, seasonings, noodles, canned foods, etc.), because the food products are industrially produced, the products likely contain ingredients that differ from natural food products, and are therefore preferably discriminated from natural food products. In these cases, it is conceivable that the lifestyle factor information acquisition unit could be configured to acquire information attached to these food products in advance through product codes such as barcodes, numbers and QR codes (registered trademark), and to acquire these ingredients retained in advance using the codes, etc. as keys.

The “exercise data” includes, for example, information such as the type of exercise and details of the exercise, the exercise time, amount of exercise (such as weight×movement distance), quality of exercise, number of steps, amount of movement, amount of body motion, heart rate changes, posture, acceleration, exercise start time and end time, location where exercise was performed, temperature, humidity, presence or lack of a coach, and devices used in the exercise. In addition, changes in perspiration amounts, blood pressure, and blood sugar values before and after exercising are also included.

These types of data may likewise be acquired using a video camera function of a smartphone, which is a lifestyle factor information acquisition unit, and analyzed using image recognition technology to analyze the video, and likewise, may be analyzed and acquired through a voice recognition function and a microphone, which is a lifestyle factor information acquisition unit, and likewise, can be selected from a pull-down menu, which is a lifestyle factor information acquisition unit, and acquired. These types of exercise data may also be acquired through communication between other exercise tools and a smartphone or the like. In addition, acquisition of exercise data can be realized by adding a communication function to training machines at a gym. Other functions such as a pedometer, heart rate monitor (in some cases, the heart rate is measured using a smartphone light and camera), and GPS provided on a smartphone or the like may also be used.

“Sleep data” includes, for example, the bedtime, falling asleep time, awakening time, getting out of bed time, number of hours of sleep, the sleep depth, awakening during sleep, amount of sleep, sleep debt, sleep phase changes, sleep latency, respiration, body movement, snoring, and lighting conditions when not sleeping and during sleep. The lighting conditions when not sleeping and during sleep include, for example, the transition in ambient lighting, blue light and other such light spectrums and amounts, and the number of hours a person is basked in light. Sleep data also includes, for example whether or not a person gets up when sleeping to use the bathroom and the time and details of such, whether or not a person ingests sleep aids, the ingestion time and amount, and the sleep rhythm and quality.

For the acquisition of these pieces of data, values measured using a brain wave measurement device are acquired through a smartphone or other such communication unit (through Bluetooth (registered trademark)) that functions as a lifestyle factor information acquisition unit. In addition, information such as the loudness of snoring and the continuation time may be acquired through a smartphone microphone that functions as a lifestyle factor information acquisition unit, and information such as the number of times that a person turns over while sleeping may be measured based on contact sound between the bedding and pajamas. Furthermore, a device such as a sleep measurement device sold by Omron Corporation may be used to acquire information through a communication unit (Bluetooth (registered trademark) for example) that functions as a lifestyle factor information acquisition unit.

“Stress data” includes the following data as autonomic nerves stress related biological information, and can be divided into non-invasive data and invasive data. Non-invasive data includes, for example, data on small changes in heart rate, electrical resistance of the skin and changes thereof, pupil diameter and changes thereof, the organic acid value in urine and saliva, hormone values and the metabolites thereof and changes within a given day, and as basic biological information, changes in blood pressure, and changes in body temperature. Examples of invasive data include, for example, changes in blood sugar.

As stress data, voice emotion recognition technology for example can be used, and changes in pathology based on the voice can be recorded through the ordinary use of a portable terminal. Voice emotion recognition technology is technology that can easily confirm by eye the percentage of “anger”, “joy”, “sadness”, and “normal” (four emotions) contained in the voice and the extent of “excitement”, and with this technology, the pathology of a patient is determined in the same manner that friends or family understand a person's state of health through the person's voice, and conditions of depression or stress can be evaluated. Furthermore, conditions of depression and stress can be evaluated with an optical topography examination. An optical topography examination uses near infrared light, which is minimally found outside, on the body to measure blood flow in the brain and to thereby numerically understand the conditions of depression. Stress data can also be acquired by using the video function of a camera of a smartphone or other such lifestyle factor information acquisition unit and the lighting of the smartphone or the like to measure the speed at which pupils dilate and contract.

Furthermore, stress data also includes the voice print and changes thereof, bathing conditions, mood, and symptoms of self-consciousness such as fatigue and dullness. Bathing conditions include, for example, the bathing time, the bathing environment such as whether the person uses a bathtub or shower (including the bathing posture), the water temperature, the bathing time, the time required for bathing, and changes in body temperature, heart rate, and oxygen saturation. Stress data may also include, for example, relaxation, hobbies, travel, SNS, meditation, spiritualness and other such mental activities, and, for example, the social status with regard to family, place of work, friends, and community, etc.

These types of information can be acquired using recognition items and the video function of a camera of a smartphone or other such lifestyle factor information acquisition unit, and likewise, can be acquired by selecting from a pull-down menu, which is a lifestyle factor information acquisition unit.

Note that in addition to these, stress data may also include the amount of perspiration, the number of hours of continuously gazing at a display, the number of hours of continuous operation of an automobile, truck, airplane, boat, train, on-premise transport vehicle, or plant vehicle, etc. and/or the total number of hours of operation within a prescribed time, number of hours of continuous physical labor, number of hours of continuous assembly operations within a factory, and the like. Stress data can also be obtained by performing prescribed computations based on these.

“Biological data” includes, for example, body composition data and in vivo nutrition metabolism information. Body composition data includes, for example, body weight, height, and body fat. In vivo nutrition metabolism information includes, for example, the number of times of urination and stool passage, the amount, the characteristics, and the coloring. Biological data also includes, for example, nutritional components, hormones, minerals, vitamins, organic acids, harmful substances, bacteria types and amounts, and the pH and the like found in hair, fingernails, blood, urine, stools, and other biological substances and discharged substances. In addition, information obtained by a technique of predicting food product components in tissues through a laser reflection characteristics analysis of the skin can also be acquired as biological data.

In addition, for example, data such as blood pressure, heart rate, oxygen concentration in the blood, the acid-base equilibrium of blood, and various inflammation marker CRPs and NF-κB are also included in the biological data.

As biological data for evaluating cancer, for example, data from image examinations such as CT and MRI data can be acquired as biological data. Cancer is usually detected through image diagnostic based examinations, and ordinarily, the progression thereafter is evaluated using diagnostic imaging. However, for example, with early stage cancer that cannot be captured in images, the evaluation data of circulating tumor cells (CTCs), which are cancer cells that appear in the blood, can be acquired as biological data. Furthermore, genetic information obtained by sampling peripheral blood or intraoral cells in saliva, and gene expression such as the presence or lack of cancer related genetic markers obtained by sampling peripheral blood are also types of biological data. Tumor marker values and inflammation marker values can also be used as biological data.

Biological data can be acquired from devices such as a “body weight scale”, “height scale”, “lung capacity measurement device”, “body composition system”, “CT device”, “MRI device”, “PET diagnostic device”, “X-ray filming device”, “ultrasound diagnostic device”, “bone density measurement device”, “heart rate measurement device,” “blood analysis device” “urine analysis device”, “saliva analysis device”, “stool analysis device”, “visual acuity measurement device”, “sweat analysis device”, “hair analysis device”, “endoscope device, “body temperature measurement device”, “hearing capability measurement device”, and a “perspiration amount measurement device” using the communication function of a smartphone or other such lifestyle factor information acquisition unit. Likewise, the data thereof can be selected and acquired from a pull-down menu that is a lifestyle factor information acquisition unit.

The “lifestyle factor information accumulation unit” has a function of accumulating the acquired lifestyle factor information as history information. The accumulation time period can be set, as appropriate, according to device performance and the like, but the ability to accumulate on a yearly unit is preferable because there is significance in making comparisons with the same season the previous year. Furthermore, conceivable intervals at which lifestyle factor information is accumulated include 1 second, 10 seconds, 1 minute, 5 minutes, 30 minutes, 60 minutes, 120 minutes, 240 minutes, and other such intervals, and the accumulation interval may be differed according to the lifestyle factor information to be accumulated. For example, the blood pulse is accumulated at a unit of 1 second, but food and beverage data may be accumulated at a unit of 120 minutes, and the number of hours of sleep may be accumulated at a unit of 240 minutes.

“History information” is desirably accumulated as history information for which the acquired lifestyle factor information is associated with the acquisition time. Here, in some cases, lifestyle factor information of a specific one time only is called history information, and in some cases, lifestyle factor information of two or more times, or of a plurality of times is called history information. Conceivable types of lifestyle factor information include information of a plurality of times that is averaged and used, or an increasing trend or a decreasing trend of numeric values can be used. This is because a decision that is made based on only lifestyle factor information of a single time can be slightly off in some cases. If lifestyle factor information is acquired as a numeric value, then with regard to, for example, the number of heart beats, body temperature, blood pressure, BMI, abdominal girth, visceral fat, body fat percentage, visual acuity, ocular fundus internal pressure, lung capacity, hearing capability, urine proteins, urine sugar, urine ketone bodies, occult blood in urine, urobilinogen, specific gravity of urine, urinary bacteria, blood total bilirubin, blood ZTT, blood TTT, blood ALP, blood CK (CPL), blood LAP, blood LDH, blood γ-GTP, blood cholinesterase, blood AST, blood ALT, blood total cholesterol, blood neutral fats, blood HDL cholesterol, blood LDL cholesterol, serum amylase, blood uric acid, blood urea nitrogen, blood total protein, blood serum albumin, blood protein A/G ratio, protein fractionation, blood creatinine, blood eGFR, blood TSH, blood serum iron, blood BNP, blood sodium, blood potassium, blood calcium, blood chlorine, blood inorganic phosphorous, blood total homocysteine, serum CRP, serum RF quantification, serum ASO, serum LOX-index, syphilis serum reaction, serum HBs antigens, serum HCV antibodies, blood cell count, hemoglobin/hematocrit, neutrophils, lymphocytes, eosinophils, monocytes, basophils, and as tumor markers, CEA, α-FP, Ca19-9, CA125 (female), CA15-3 (female), PSA (male), gastrin-releasing peptide precursor, cytokeratin 19 fragments, SCC, NSE, STN, blood glucose, insulin, hemoglobin A1c, pepsinogen 1.2, center of gravity sway examination, blood pressure pulse wave, bone mass measurements, female hormone FSH, female hormone estradiol, facial color, fingernail color, color of the back of the eyelid, and the like, values such as the average value, dispersion, deviation value of data each time, t-distribution, F-distribution, and chi-squared distribution may be determined (statistical processing means) on the basis of past numeric values, advice output based on past values thereof may be recorded, and the results of the advice may be statistically determined (advice evaluation means). Therefore, the advice selection unit may have a statistical selection means so that advice with the greatest potential for reaping results from the advice is selected. Furthermore, correlation coefficients of each of these lifestyle factor information items are determined (correlation coefficient calculation unit), and for lifestyle factor information items having a correlation extent of a certain level of higher, for example, 0.7 or higher, an advice selection rule change unit that changes the advice selection rules so that these lifestyle factor information items are grouped together and a single advice item is selected may be provided. The same also applies to cases for which items having a high correlation coefficient are in a relationship in terms of cause and results. Processing for selecting advice based on this type of detailed data processing has not been performed in the past, and as the data volume and data types (lifestyle factor information here) become more diverse, a problem of mutual inconsistencies arising with simple advice selection has existed, but this can be resolved with the present invention. With regard to lifestyle factor information for which the correlation is thought to be high in advance, as described above, the present invention may be configured to narrow the advice down to a single piece of advice using an evaluation function (function for evaluating a plurality of lifestyle factor information items) that uses a plurality of related lifestyle factor information items and lifestyle factor information optimum values corresponding to each item thereof as a plurality of group variables. Conventional advice devices also do not carry out this type of processing.

The present device may also be configured so as to communicate with a statistical processing server, and may be configured to collect a history of lifestyle factor information for each user from the present device in Japan and in other areas throughout the world. The present device may also be configured so as to collect advice information for which the history of lifestyle factor information and the time axis are synchronized and output. Correlation coefficients can be calculated (correlation coefficient calculation unit) through so-called big data processing, and through this, correlation coefficients of lifestyle factor information items themselves can be obtained with the correlation coefficients being more reliable than users independently viewing the correlation of lifestyle factor information. In addition, the relationship between the lifestyle factor information and the advice that is output is also the same. Therefore, through cancer prevention, recurrence prevention, and improvements, effective advice can be determined by statistical processing. It is also possible to discover new cancer risk parameters by using statistical processing and artificial intelligence (AI) processing. In other words, when the correlation coefficient is high, there may be cases in which the results are based on a cause for which these lifestyle factor information items are common, and there may be cases in which these are in a relationship between the cause and result. With regard to whether a potential exists for either of these relationships to hold true as data, processing to associate identification information with each lifestyle factor information item is also conceivable. If this is done, the discovery of new cancer risk parameters becomes possible with high reliability. Furthermore, feedback on correlation coefficients of lifestyle factor information items themselves, estimations of the relationship between a cause and a results, estimation of a result on the basis of a common cause, and effective advice are provided from the server to each of the present devices, or the information is usably retained and accumulated as data, and each of the present devices can be operated so as to output more highly effective advice. This is the creation of unknown information on the basis of so-called big data processing. Note that a central server and the present device may be configured so as to be able to add, change, and delete processing to output new advice information to each of the present devices from this type of central server, add the information to the advice information retaining unit, and delete useless advice or advice with little effect that is retained in the advice information retaining unit, and advice selection rules retained in the advice selection rule retaining unit in association with these processes. In addition, if the present device is configured so as to communicate with a statistical processing server, and is configured to collect a history of lifestyle factor information for each user from the present device in Japan and other areas throughout the world, the present device is preferably configured so as to transmit the history information along with user attributes. Here, user attributes include residential area (name of country, name of region), type of occupation, form of labor, gender, age, and race, etc. Cancer is said to have regional characteristics, and is said to exhibit dependence on occupation type, form of labor, gender, age, and race, etc. Accordingly, it is thought that more effective advice information can be output and correlations between lifestyle factor information items themselves can be discovered by analyzing according to each of these attributes rather than merely analyzing big data as is. Of course, the transmitted data is associated with time information, and preferably, consideration can also be given to the dependence on the country, region, and season. Furthermore, for cases in which a unique accident has occurred such as the Fukushima Daiichi Nuclear Power Plant accident, that information is also preferably included in the attribute data. This includes particularly information relating to radiation exposure from accidents such as nuclear power plant accidents, nuclear power plant malfunctions, accidents and malfunctions at radioactive waste treatment facilities, and accidents and breakdowns in radioactive waste transport.

The “lifestyle factor information optimum value retaining unit” has a function of retaining lifestyle factor information optimum values, which are optimum values for each lifestyle factor information item directed at the purpose of cancer prevention and improvement. Note that the lifestyle factor information optimum value may correspond to lifestyle factor information that is a combination of a plurality of lifestyle factor information items. Furthermore, the lifestyle factor information optimum value has a prescribed width, and the present invention may be configured such that the presence of an optimum state is determined when the lifestyle factor information falls within the prescribed width.

The “lifestyle factor information optimum values” are optimum values for each of the lifestyle factors directed at the purpose of cancer prevention and improvement. “Lifestyle factor optimum values” are optimum values of various lifestyle factors such as, for example, meal information, exercise data, sleep data, stress data, and biological data. For the optimum value of each lifestyle factor, the condition of each user is registered so that the optimum value thereof can be preferably set as an optimum value that differs depending on factors such as age and gender, as is the case with blood pressure for example. A conceivable method for determining an optimum value is to statistically process the lifestyle factor information of healthy people to find the average value, and then set a value range (±σ for example) of a prescribed width from the average value. Note that the precision is increased by determining optimum values using values not only for healthy individuals but also for individuals suffering from cancer. In other words, because the reliability of an optimum value can be measured, an optimum value can be used as an optimum value with reliability. It is conceivable that optimum values for individuals suffering with cancer can also be set as values that are unique to cancer patients. Compared to healthy individuals, in some cases it may be preferable for a cancer patients to have, for example, a pH value of urine that is shifted further to the alkaline side.

The “advice retaining unit” has a function of retaining a plurality of advice items for more closely approaching the optimum values of each lifestyle factor information item. A plurality of advice items is retained. Plurality means a concept that includes both a case in which multiple advice items are retained for a single lifestyle factor information, and a case in which multiple advice items are retained for multiple lifestyle factor information items. The reason for designing to include a plurality of advice items is to enable the selection of the most suited advice according to the level of separation from the lifestyle factor information optimum value. The advice output method of conventional devices is that of outputting advice by choosing from two alternatives, namely whether a condition is satisfied or not satisfied, but with this style, in some cases the user is not provided with sufficient motivation. In other words, even if a user's condition is significantly separated from the lifestyle factor information optimum value, rather than conveying that information in a straightforward manner to the user, in some cases it may be better to provide encouragement with some sugarcoating of the details. The intention of this matter is to more closely approach a scene in which the user is verbally receiving advice from a specialist. Specialists try to maximize the effectiveness of medical treatments, etc. by providing encouragement and a vigorous pep talk to the user while inferring the user's facial countenance. However, with application software and specialized devices, etc., messages to be delivered are generated merely on the basis of whether a condition has been met or not, and therefore currently, such application software and devices are inferior to advice from a specialist. Accordingly, in the case of a device of the present invention, a configuration is conceivable for which, for example, a personality diagnostic test is performed, and based on that test and genetic examination data and other such information, an understanding of the personality of the user is acquired, and a plurality of types of advice is selected according to the understood personality. Furthermore, the device may also be configured so that the mood and other details of the user that day are input, and appropriate advice can be selected from an advice group that is suited for that mood. The device may also be configured so that rather than directly inputting a user's mood, the details of an email sent by the user are morphologically analyzed, the mood and ambience of the user that day are understood according to keywords appearing in the email, and appropriate advice can be selected from an advice group suited to that mood and ambience. In addition, the mood of a user on a given day may be inferred based on attribute information of webpages that were viewed, and the mood of that day may be inferred based on weather information and news information. For example, if the news for the day is news about a natural disaster, it can be inferred that the user's mood is somber, and if the user is a Hanshin fan, and the Hanshin Tigers win a game, it can be inferred that the user is in a cheerful mood. Voice emotion inference technology may also be used. This technology infers the mood and emotions of a person based on the tones of the person's voice, and for example, if voice tones are high, it is inferred that a person is stressed. Advice content is not particularly limited, and advice may be provided not only on lifestyle habits such as meals, exercise, sleep and bathing, but also on items such as supplements.

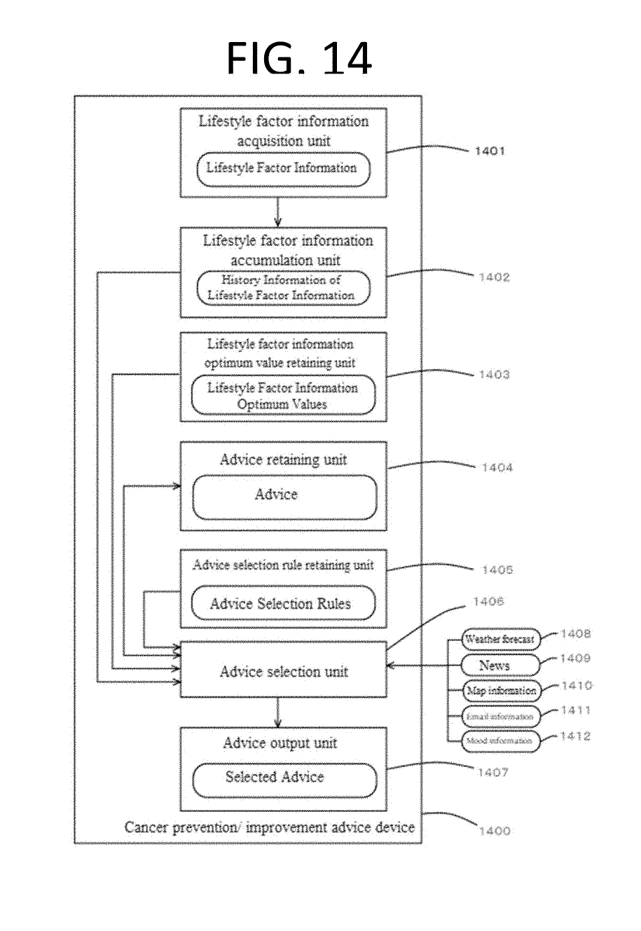

FIG. 14 is an image showing a functional block of the present invention for acquiring various information through a network in order to select advice. With the advice selection unit, for example, weather forecasts, news, map information (with GPS data), email information, and mood information, etc. are acquired, and in order to take into account these external information items and to select advice, advice is selected in accordance with advice selection rules retained in the advice selection rule retaining unit.

The advice selection rules are not limited to a form of direct selection on the basis information such as the level of divergence from lifestyle factor information optimum values and acquired lifestyle factor information, a different value, and a ratio. For example, rules may be in a table usage format in which one or more advice item is selected from a plurality of advice items according to the lifestyle factor information values. If ten (a plurality of) advice items can be selected for one lifestyle factor information item for example, the rules may be configured such that the advice item is randomly selected, or advice is selected based on the relationship with other lifestyle factor information, or on externally acquired information (weather, news, text contained in emails, the “like” mark, mood selections, information from GPS, and geographic information).

### <Advice Regarding Carbohydrate Restriction>

If, for example, an advice item pertains to carbohydrate restriction, then advice such as “How about ramen today for the first time in a while,” “Would you like spaghetti today,” “There is a restaurant nearby that has a salad bar,” “Did you eat too much for lunch today,” “You're going out for drinks tonight, so try to eat only a light lunch,” “Your state of health doesn't seem to be good, be sure to eat plenty of meat,” “Try to be moderate in alcohol consumption today,” “Sashimi would be great for dinner today,” and “Today's recommendation is grilled fish” can be provided. These advice items nonchalantly encourage carbohydrate restriction in a form of proposals without directly mentioning numeric values of lifestyle factor information. Numerous types of these advice items may be prepared, and for example, the configuration may be such that instead of selecting the advice of “You're going out for drinks tonight, so try to eat only a light lunch,” the advice of “Try to eat sashimi for lunch so that you can eat lots tonight at the drinking party” can be selected. When the same advice is always given, the user could lose interest and begin to feel that the application is blunt, and therefore, for example, as with the Tamagotchi (registered trademark) product that was previously a major fad, the preparation of various types of advice according to the situation creates a warmer feel, and can provide users with motivation to optimize their lifestyle factor information. Furthermore, in addition to preparing advice in advance, a plurality of portions can be prepared, and those portions can be combined, as appropriate, to thereby configure advice. For example, the design could be such that “sashimi”, “tofu cuisine”, “yogurt”, “cheese”, and “eggs” can be registered as word groups that are handled in the same line, and the abovementioned advice of “Try to eat sashimi for lunch so that you can eat lots tonight at the drinking party” can be changed in some cases to “Try to eat tofu cuisine for lunch so that you can eat lots tonight at the drinking parts.” When such changes are made depending on the case, the advice may be appropriately designed using predetermined rules, or may be designed to be selected with merely a random number, and a selection algorithm may be adopted so that there is no overlap with the previous two times. The scope of advice is not limited to menu details, and may include details such as a restaurant that should be visited, ingredients that should be purchased and service recommendations. When carbohydrates are being restricted, curry rice cannot be recommended, but if a user desires curry rice, advice can be given such as “Let's try coconut milk curry with turmeric that contains curcumin, which has anticancer activity, and for the rice, let's try diet rice, which is low in carbohydrates. This meal is rich in medium chain fatty acids found in residues that increase the absorption of curcumin, which has a poor absorption rate.”

### <Advice Relating to Exercise>

Examples of advice relating to exercise include the following. “After today's meal, you're going to feel tired in about 30 minutes, so how about going for a light walk?” “Today's weather is good, so how about walking to the next train station?” “It's rainy today, so how about walking in underground passages?” “It looks like you have free time today. Consider visiting the nearby tourist spots.” “The Kasai Rinkai Park isn't crowded today. How about taking a walk through the aquarium and park?” “Will you help the kids practice jumping rope?” “The sports gym is holding a weight training practice today. Let's participate!” Note that as is understood from the examples given above, current location information may be obtained by GPS and used in the advice, information from a scheduler (scheduling software) incorporated in a smartphone or other device may be used, and the news and weather forecast for the given day may be obtained and used in the advice by automatically connecting to a network.

<Advice Relating to Sleep>

Examples of advice items relating to sleep include the following. “Around seven hours of sleep is ideal. That's a good rhythm.” “Your sleep rhythm is good. Meal rhythm is also important for keeping your biological clock functioning properly.” “It's good to use indirect lightning at night.” “It's best to dim the lights thirty minutes to one hour before sleeping.” “Once you wake up in the morning, bask in the daylight! The light will reset the biological clock for your brain and nervous system.” “Try to stop eating three hours before sleeping.” “Try to avoid looking at blue light in the nighttime hours.” “Your number of hours of sleep tends to be slightly long. Be careful because sleeping too much can increase your risk for obesity.” “A bath may help when you're having difficulty falling asleep.” “When your lifestyle rhythm is disrupted by staying up late and sleeping in, it's easier to gain weight.” “You tossed and turned a lot while sleeping. Did you eat a carbohydrate-heavy meal before sleeping?” “Maybe you should try using a new pillow.” “You coughed a lot while in bed. Dry air might be the cause. Maybe you should try using a humidifier.”

### <Advice Relating to Stress>

Examples of advice items relating to stress are as follows. “Let's try relaxing with some good quality sleep.” “Taking a bath is known to have a calming effect on your mood.” “Maybe you should take a trip to the northeast. The fall colors are beautiful this time of year. You've been very busy at work recently, so maybe you should try traveling occasionally.” “There is a highly recommended karaoke club nearby. You can get a reservation right now.” “This is a great season for golf. How about getting a little exercise on an easier short course?” “It seems that you are not getting enough sleep. The ideal amount of sleep is around seven hours.” “If you're feeling irritated, try taking a deep breath.” “You have a golf reservation next week. Let's send your clubs by express delivery by at least two days before your reservation.” “Your popularity on SNS is good.” “There is a spiritual book that is being recommended. Shall I introduce you to it?” “It looks like you liked your birthday present. Your girlfriend posted about it on SNS.” “Good job on organizing the company barbecue today. Everyone had a great time.” “Next week you're going out for drinks with some old college friends. Maybe you should dress in something fashionable. Should I introduce you to a site that is having a sell on dresses?” “Your Mother's Day present last year was a major success, and your mother even sent you a photo of it from her cell phone. How about giving her flowers this year?”

### <Advice Relating to Biological Data>

With the present invention, the pH level of urine is preferably acquired as one type of biological data. This is because for cancer prevention and improvement, it has been shown that body fluids are preferably alkaline. Acidic environments are microenvironments that are advantageous for cancer cell activity, and in order to realize this type of acidic environment, cancer cells transport lactic acid to the outside of cells. Sodium is necessary for that transport, and it is thought that restricting sodium hinders the development of microenvironments that are suited for cancer cells. Therefore, if the pH of urine can be obtained, and advice can be received, such advice can lead to cancer prevention and improvements. Examples of advice relating to urine pH in order from poor to optimum conditions are as follows. “Your urine pH is low, and your body fluids are tending to be acidic. Try to reduce your protein intake, and eat more vegetables and foods containing potassium. Also try to drink less alcohol.” “Your urine pH is at an optimum value for cancer prevention and improvement. Your body fluids are being maintained at alkaline levels, and are optimal values. You're making favorable improvements in your meal and lifestyle habits for the purpose of cancer prevention and improvements.”

With the present invention, the ratio of sodium to potassium in the urine is preferably obtained as one type of biological data. Restricting sodium hinders the creation of microenvironments that are suited for hosting cancer cells, and therefore restricting salt content and consuming potassium that discharges salt content is effective, and a high ratio of sodium to potassium in the urine is not favorable. This is because an amount of sodium to an amount of potassium of 1 or less is effective for cancer prevention and improvement. Examples of advice regarding the ratio of sodium to potassium in the urine in order from poor to optimum conditions are as follows. “Your sodium value is high. Try to eat more vegetables and foods containing potassium.” “Your sodium value is slightly high. Try to eat less salt.” “Your potassium and sodium values are good.”

### <Advice Based on Lifestyle Factor Information Across a Plurality of Types of Categories>

With respect to cancer prevention and improvement, glucose metabolism (changes in blood sugar) provides energy that is preferentially used by cancer cells, and when carbohydrates are restricted, in many cases, cancer growth is directly reduced. In addition, the representative elements of lifestyle factor information including meal information, exercise data, sleep data, and stress data, and all of the biological data items are mutually involved, and are important factors for advancing with cancer prevention and improvement. Namely, external factors that are involved include the amount and type of sugar that is consumed, and the metabolism of sugar through exercise, and internal factors that are involved include autonomic nerves, the secretion of hormones (primarily insulin), and nutrition conditions. The details will be discussed below.

If signs of ischemic heart disease are suspected from an overall determination based on lifestyle factor information, a configuration is conceivable in which options are increased in order from the lowest severity from the following advice group and output as advice. For example, if the severity is at a minimum level, the advice could be merely “Try not to increase your body weight,” and in a case of the next lowest level of severity, the configuration could be such that two advice items are output including “Try not to increase your body weight” and “Try to increase your intake of dietary fiber from seaweeds (wakame, hijiki, kombu, etc.) and vegetables.” Examples of advice are as follows.

“Try not to increase your body weight.” “Try to increase your intake of dietary fiber from seaweeds (wakame, hijiki, kombu, etc.) and vegetables.” “As a general rule, consuming excessive alcohol is prohibited. If you're going to drink, limit your drinking to 90 ml of Japanese sake or one-half a cup of shochu or one sake cup of double whiskey or 250 ml of beer, and avoid drinking on consecutive days.” “Try to eat the right foods so that you get enough dietary fiber (vegetables, wakame, mushrooms).” “Try not to eat too much salt. Eat less Japanese pickled vegetables and remember that foods such as soy sauce, sauces, and miso contain salt. One gram of salt ⅕ a teaspoon.” “Tobacco makes diseases worse, so try not to use tobacco.” “Refrain from eating sweets.” “Try to walk more as a measure to alleviate stress.”

If signs of hyperlipidemia are suspected from an overall determination based on lifestyle factor information, a configuration is conceivable in which, for example, options are increased in order from the lowest severity from the following advice group and output as advice.

“Aim for well-balanced eating habits.” “Pay more attention to your habit of eating high calorie meals and overeating. Pay particular attention to rice, mochi, bread and noodles, and try not to eat too much of these types of foods.” “Add 10 g/day of linseed oil, which contains omega-3 fatty acids, to your diet.” “Let's reduce your intake of milk products (milk, butter, cheese, fresh cream, etc.).” “Try not to consume too much salt, sugar, confectioneries, and milk products. Vegetables, seaweed and mushrooms are rich in dietary fiber, and suppress cholesterol absorption, so try to consume a sufficient amount of these foods.” “Refrain from using tobacco in order to reduce oxidation stress.” “Let's overcome your lack of exercise by jogging or walking at a rapid pace for 3 km/day.”

If signs of high blood pressure are suspected from an overall determination based on lifestyle factor information, a configuration is conceivable in which, for example, options are increased in order from the lowest severity from the following advice group and output as advice.

“Avoid overworking and a shortage of sleep, and try to maintain a lifestyle with correct principles.” “Let's avoid extreme changes in air temperature.” “Let's avoid intense exercise, and continue with light exercises using your legs.” “Refrain from coffee and tobacco.” “Set your bath temperature to around 40 to 41 degrees, and soak in the tub up to your shoulders for 15 minutes or longer.” “Try having a bowel movement.” “Blood pressure can be controlled in some cases by merely reducing salt intake, so let's trying reducing your salt intake to 5 g or less per day. Also cut back on processed foods, salted foods, Japanese picked vegetables, and miso, soy sauce and salt used in cooking.” “If you're obese, let's try restricting carbohydrates.” “Let's reduce carbohydrates and fats. Try to proactively eat more soybeans and soy products.” “Try to proactively ingest more potassium (from vegetables and fruits) and calcium (from milk, milk products, small fish, tofu, and green and yellow vegetables). Be certain to get enough potassium especially when taking a hypotensive diuretic.” “Try to ingest dietary fiber from seaweeds (wakame, hijiki, kombu, etc.), vegetables, and fruits.” “Excessive alcohol is prohibited. Let's limit alcohol intake to 0.18 liters of Japanese sake or one can of beer or around three sake cups of whiskey.”

If after giving advice to reduce the intake of dietary salt, the signs of high blood pressure are thought to be high from an overall determination based on lifestyle factor information, a configuration is conceivable in which, for example, the number of advice options is increased in order from one according to severity, and advice is output from the following advice group.

“Aim for a balance of flavors while cutting back on salt content and sugar content.” “Rather than dividing your dietary salt a little at a time, let's add it as an accent to one meal.” “Let's use fresh ingredients and fully capitalize on the inherent flavors of those ingredients.” “Let's use the natural good flavors of kombu, wakame, mushrooms, and dried bonito.” “Let's use the sourness of yuzu, bitter orange, and lemons, etc. and spices.” “Try to apply soy sauce to only a portion of your sushi.” “How about a pot of seafood and vegetable stew and other foods boiled in water and served with dipping sauces such as ponzu and other condiments.”

If signs of obesity are suspected from an overall determination based on lifestyle factor information, a configuration is conceivable in which, for example, options are increased in order from the lowest severity from the following advice group and output as advice.

“For proteins, try to avoid meats with a lot of fat, cheeses, ham, bacon and other processed foods, and instead try to eat tofu, fish with white flesh, chicken tenderloin, beef and pork tenderloin, and thighs.” “Try to eat beef that was raised on grass.” “Try to heat grass-fed lamb.” “Avoid fatty cuisines (such as tempura, fried foods, deep-fired foods, etc.) as much as possible, and instead focus on foods cooked by boiling or stewing and steamed foods.” “Proactively consume low calorie foods that have an action of delaying fat and lipid absorption such as vegetables, mushrooms and seaweeds.” “Avoid dressings and mayonnaise.” “Use sauces, ponzu, and ginger soy sauce instead.” “Try to correct unfavorable meal patterns such as consuming irregular meals, excessive eating, unbalanced eating, snacking all the time, and eating a lot at night.”

Also, if it is determined that the severity is at a high level, advice for cutting carbohydrates could be output in stages. Examples are as follows. “Let's limit the amount of rice that you eat to two cups per day.” “Let's limit the amount of rice that you eat to one cup per day.” “Let's limit the amount of rice that you eat to half a cup per day.” “Let's refrain from eating rice.” “In addition to refraining from eating rice, let's also cut back on the amount of root crop based foods that you eat.” “Let's refrain from eating rice and noodles.” “Let's refrain from eating rice, noodles and flour based foods.” “Let's refrain from eating rice, noodles, and flour based foods, as well as tempura.” “Let's refrain from eating rice, noodles, and flour based foods, and also from consuming sugars.”

A determination of “obesity” may be made in a manner such as follows. Namely, body weight and height are used as lifestyle factor information, and the BMI is calculated as a lifestyle factor information item. The equation is as follows.

BMI=body weight (kg)÷(height (m)×height (m)). If the BMI is 25 or greater, as this numeric value becomes larger, the advice listed above is configured so that device described at the later end is selected in stages. The determination of “obesity” can also be made using the amount of body fat, the body fat percentage, and the lean body mass as determination indicators.

The “advice selection rule retaining unit” has a function of retaining advice selection rules, which are rules for selecting the retained advice using the history information of lifestyle factor information and the lifestyle factor information optimum values retained for those lifestyle factors as variables.

The “advice selection rules” are rules for selecting the retained advice using the history information of lifestyle factor information and the lifestyle factor information optimum values retained for those lifestyle factors as variables. These rules are basically rules for selecting appropriate advice according to the extent of divergence between the lifestyle factor information that has been obtained and accumulated and the lifestyle factor information optimum values, which are optimum values for those lifestyle factors. These rules are rules for selecting advice so that narrowing the divergence according to the state of divergence, and achieving goals becomes enjoyable. Therefore, each of the plurality of advice items is tied to the state of divergence. If the extent of divergence is large, advice is linked so that strong motivation can be imparted, and if the extent of divergence is small, advice is linked so as to comfortably cause that type of behavior to occur. However, ordinarily, the provision of firm advice when the divergence extent is large has the opposite effect, and therefore advice that imparts motivation without the user understanding such is favorable.

Using the recently given example as an example, if the divergence is small, advice that instead goes against the carbohydrate restriction is given such as “How about ramen today for the first time in a while,” or “Would you like spaghetti today?” Meanwhile, if a certain level of divergence is recognized, advice such as “There is a restaurant nearby that has a salad bar,” “Did you eat too much for lunch today,” “You're going out for drinks tonight, so try to eat only a light lunch,” “Your state of health doesn't seem to be good, be sure to eat plenty of meat” is given. If the divergence is large, advice is provided such as “Try to be moderate in alcohol consumption today,” “Sashimi would be great for dinner today,” and “Today's recommendation is grilled fish” can be provided.” Note that if advice such as “Sashimi would be great for dinner today” is given, it is conceivable that the user could be guided to a nearby restaurant selling sashimi based on GPS data, and leaflet information for that day could be further provided. This type of service is possible on a person-to-person basis, but conventional devices have not provided this type of service, and therefore the present invention feels like a friendlier, warmer device. In addition, the reliability of the device is further increased by enabling the device to be sympathetic, and motivation to achieve goals is thereby increased. Note that divergence may be a difference between lifestyle factor information and lifestyle factor information optimum values, may be a ratio with lifestyle factor information optimum values, or may be data for which these have been weighted prescribed amounts. Weighting may be designed so as to change daily. Changes in weighting may be stipulated based on the relationship with other lifestyle factor information items, and may be changed according to the inferred mood and ambience of that day. Weighting may also be changed according to circumstances such as a user is already suffering from cancer, or a person was treated for cancer and is in remission, and is now trying to prevent recurrence.

The “advice selection unit” is configured so that advice is selected using the retained advice selection rules with the history information of the lifestyle factor information and the lifestyle factor information optimum values retained for those lifestyle factor items being used as variables. The advice selection rules can be changed similar to the optimum values for the lifestyle factors. For example, if biological data for which the expression of CTCs, or in other words, circulating cancer cells in the blood, has been recognized is acquired as lifestyle factor information, or if biological data for which the expression of cancer related genes has been recognized is acquired as lifestyle factor information, setting the optimum values for lifestyle factors and the advice selection rules to stricter conditions is desirable. This is because aiming for cancer improvements by increasing the quality and speed of lifestyle improvements is anticipated.

The present device can use advice selection rules to select advice using the history information of singular lifestyle factor information accumulated by the lifestyle factor information accumulation unit and a singular lifestyle factor information optimum value as variables. The present device can also use advice selection rules to select advice using the history information of a plurality of lifestyle factor information items accumulated by the lifestyle factor information accumulation unit and a singular lifestyle factor information optimum value as variables. Furthermore, the present can also use advice selection rules to select advice using the history information of a plurality of lifestyle factor information items accumulated by the lifestyle factor information accumulation unit and a plurality of lifestyle factor information optimum value as variables.

The “advice output unit” is configured so as to output the advice selected by the selection unit. The output may be performed, for example, with text, images (video and/or still images), and graphics, etc. on a television or smartphone display, or may be performed through audio through a speaker or earphones. In addition, the output may be performed using a URL or QR code (registered trademark) that contains URL information to indicate a location on a network server or the like where text, images (video and/or still images), and graphics, etc. can be found. Furthermore, an animated idol or animal or other such avatar may be used to speak on a screen, advice may be given in the form of a cartoon, and advice may be given in the form of a video or game. Stress has a negative impact on cancer, and therefore such measures can be adopted in order to alleviate stress as much as possible. Furthermore, the media that is used for output does not need to be singular, and at certain times, the output may be on a smartphone screen, while at other times, the information may be output through a synthesized voice telephone call to the smartphone, and at other times, it may be an email from a smartphone or PC, or a cancer prevention/improvement advice device may be disposed as a server on a network, a character of the user may be registered to a social network such as LINE (registered trademark) and FACEBOOK (registered trademark), and that character may be configured so as to talk to the individual. When this is done, it can be anticipated that the user will feel as if a medical specialist is actually speaking to him or her, that a sense of familiarity will be fostered, that stress will be alleviated, and that good effects will be achieved. In order to use a plurality of types of media to convey advice to the actual person in this manner, the plurality of media and the advice output unit must be configured so as to be capable of mutually communicating. Also, the advice output unit may be provided with an output request receiving means for receiving output requests from a user. This is so that communication can also be initiated by the user, and so that the personified present device feels as if it is another person speaking to the user. Cancer patients often feel a sense of loneliness, and in order to distract the user from the feeling of loneliness, adopting this type of configuration is preferable. Therefore, if communication from the user is requested, it is not necessary to output only information relating to “cancer”, and the device may be configured so as to also be capable of outputting information such as the latest news, topics in the entertainment industry, conversation about hobbies (in this case, information on hobbies, etc. must be registered in advance in the present device), conversations about the weather, local topics, the present status of friends and families that can be obtained from SNS, and shopping information. For this type of information, as an output function means for outputting second advice information, a second advice information output means may be provided at the advice information output unit. When the present device is configured in this manner so as to be capable of outputting also information that is not directly related to cancer, it becomes difficult to differentiate from advice relating to cancer, and therefore the user can forget for a moment that he or she is suffering from cancer, and psychological resistance to the present device can be reduced. In addition, it is also preferable that the timing at which advice is output not be a fixed timing. In other words, with the exception of when an output request is made by a user, even if a condition is such that advice can be selected and output, that is, even if sufficient lifestyle factor information has been acquired, it is best not to immediately output the advice because a user will tend to be on guard for advice at that timing. For example, it is preferable to use random number data or such to determine the timing until advice is output after conditions that enable output have been met. To do this, the advice output unit is preferably provided with an output timing determination means. Likewise, a configuration is also conceivable in which random number data or the like is used, and advice purposely is not output once out of a plurality of times. This configuration is adopted in order to impart a sensation that is closer to a person. It is also conceivable that depending on the case, the present device may project the appearance of having forgotten to provide advice, and therefore is now providing advice. In other words, a phrase such as “I didn't mention this before, but . . . ” may be used. This configuration is also used to avoid the matter of the same type of advice being continuously provided, and can reduce the occurrence of the present device feeling overly persistent from the user's perspective.

Furthermore, the present device may be configured so that as part of the advice, efforts directed at improvements are actually proactively implemented such as recommending counseling (with a real person or unmanned) for recognized behavior therapy.

Various details were discussed above with regard to the advice output unit, but because the present device is an advice device that is related to “cancer”, the form of the output is particularly important, especially from the perspectives of not imparting stress on the user, providing a feeling a familiarity, and distracting the user from a sense of loneliness. This point is a major difference from other devices that are designed for the purpose of preventing and improving other illnesses.

The purpose of outputting advice relating to cancer is to convey advice information to users and to enable users to adopt measures to deal with cancer, and thus once advice information has been obtained, it is desirable to immediately output that information. Therefore, for example, if the output destination is a smartphone display, outputting in a manner that does not hinder the use of the smartphone by the user as much as possible is desirable. For example, outputting to a portion of the smartphone screen is desirable. Similarly, the advice may be displayed in a partial region of a display called a widget screen, a browser screen, or an information screen, or such. Alternatively, the configuration may be such that the present device waits for an input operation by the user before displaying advice information so that use of the smartphone by the user is not obstructed. Such a configuration can be realized, for example, by providing a button, icon, or such for displaying advice information on a smartphone or personal computer, and when the user operates the button or such, the input thereof is received, and advice information is displayed. The advice output unit can also purchase ingredients or the like and make reservations for activities based on the advice information.

### <Description of the Hardware Configuration>

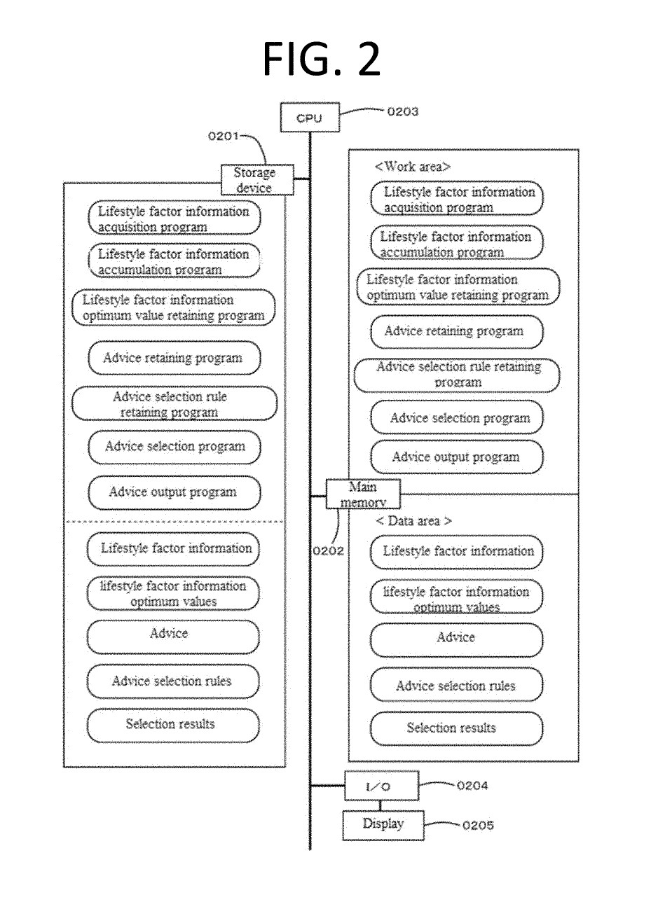

As illustrated in FIG. 2, the storage device ((0201) a hard disk or ROM device for example) stores a lifestyle factor information acquisition program, a lifestyle factor information accumulation program, a lifestyle factor information optimum value retaining program, an advice retaining program, an advice selection rule retaining program, an advice selection program, and an advice output program, and also retains, as data, lifestyle factor information, lifestyle factor information optimum values, advice, advice selection rules, and advice selection results. Furthermore, when the present device is started up, each of the abovementioned programs and necessary data are loaded from the storage device to the main memory (0202), and the present device can operate as a computer.

### <Processing Flow>

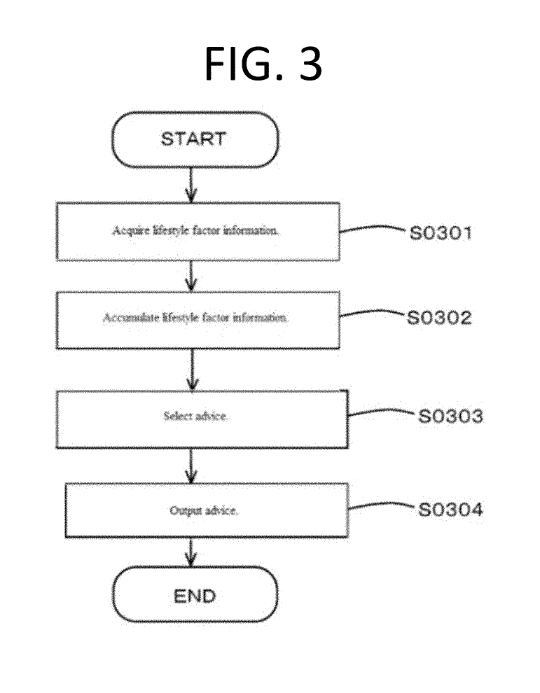

FIG. 3 shows the flow of processing by the present device. First, lifestyle factor information is acquired (S0301), the acquired lifestyle and information items thereof are accumulated (S0302), and then based on the history information of the accumulated lifestyle factor information (may be a case of a single lifestyle information, and may be a case of decryption), advice is selected in accordance with the advice selection rules (S0303). The selected advice is then output (S0304).

### Embodiment 1

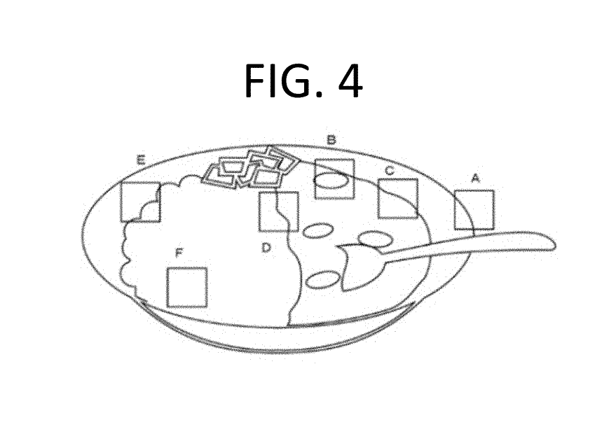

FIG. 4 illustrates a specific example for obtaining meal content (meal information) which is a lifestyle factor information detail. The lifestyle factor information acquisition unit includes, for example, a smartphone camera and image recognition function, and a recognition based food product database, and as shown in FIG. 4, when a photo of curry rice is obtained by a camera, which is a lifestyle factor information acquisition unit, the characteristic image portions (characteristic amounts) as shown by A to F in FIG. 4 are extracted and recognized, and the food product having these characteristic amounts in the recognition based food product database is identified. In this case, curry rice is generated as a food product containing these characteristic amounts. When this occurs, next, the present device accesses the food product ingredient database for curry rice, and acquires the food product ingredients for 100 grams of curry rice.

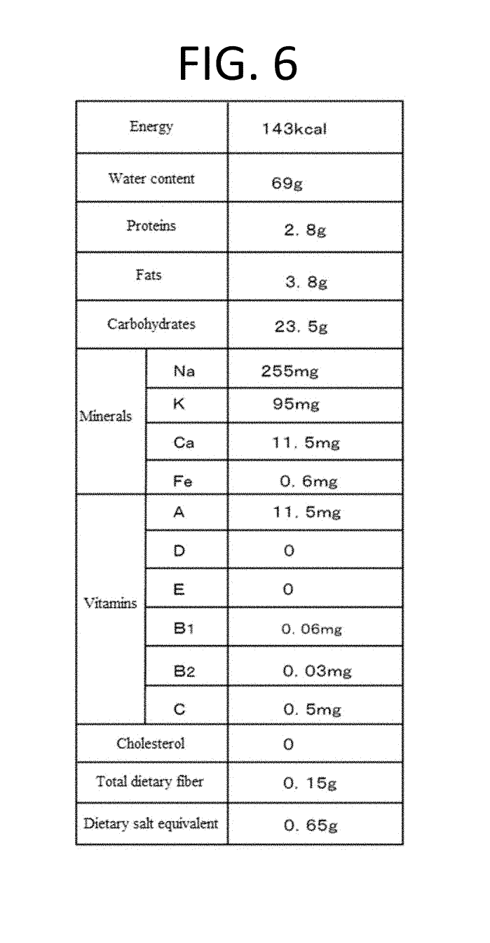

FIG. 6 shows the food product ingredients for the curry rice acquired in this manner. Likewise, image recognition technology can be used to obtain the total number of grams of curry rice, and for example, if it is assumed that the curry rice is 430 grams, the values shown in FIG. 6 are multiplied by 4.3, and the values thereof are acquired by the lifestyle factor information acquisition unit. If unclear from the image, the present device can ask the user how many grams of rice are in the meal, and the user can select and input the amount of rice.

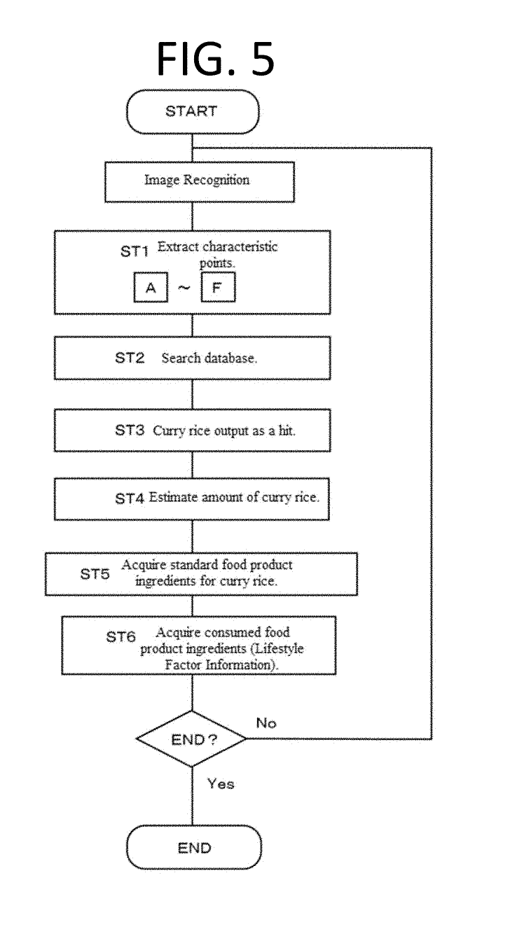

FIG. 5 illustrates this processing flow. First, image recognition is performed, next, characteristics points are extracted (ST1), a database search is performed (ST2), and the name of the food product is found (ST3). In this example, curry rice is found. Next, the amount of curry rice is estimated (ST4), food product ingredients for 100 g of curry rice and the amounts of those ingredients are acquired (ST5), and the amounts and ingredients are multiplied to acquire the food product ingredients and amounts that were consumed (ST6). In other words, the lifestyle factor information acquisition unit acquires lifestyle factor information in this manner. Lastly, the end is determined, and if it is not the end, the same processing is performed for the next food product, and when the end is reached, acquisition processing by the lifestyle factor information is terminated.

The acquired lifestyle factor information is sent to the lifestyle factor information accumulation unit and accumulated, and is used for selecting advice at the scheduled timing. For example, if a rule for determining that a user dished up too much curry rice and consumed an excessive amount of carbohydrates is retained as an advice selection rule, then for example, advice such as “For dinner tonight, refrain from eating rice, and instead, enjoy pasture fed beef that is rich in omega-3 fatty acids” is selected by the advice selection unit, and is output by the advice output unit to a screen of a smartphone for example. It is conceivable that if a user has curry rice for lunch, then advice can be scheduled to be output around 4 pm, which is when the user begins to think about dinner.

### Embodiment 2

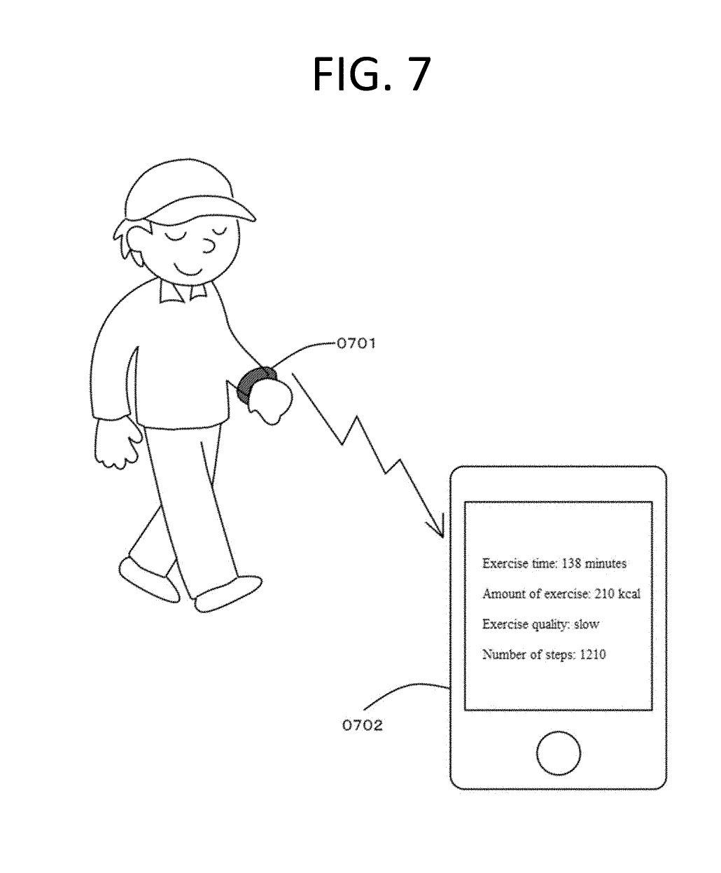

FIG. 7 shows an example of acquiring exercise data as lifestyle factor information. FIG. 7 shows conditions in which a user is walking with an exercise amount sensor (0701) strapped around the user's arm. The exercise amount sensor is equipped with an acceleration meter, a pulse rate meter, perspiration amount meter, and a GPS system. These sensors function and collect exercise data on the user through the swinging motion of the arms, and the data is transmitted to the lifestyle factor information acquisition unit of a smartphone using a communication function. The lifestyle factor information acquisition unit of the cancer prevention/improvement advice device (0702) of the smartphone transmits the acquired exercise data as lifestyle factor information to the lifestyle factor information accumulation unit, and is configured so as to display the most recent exercise data on the smartphone screen. In this example, the display shows that the exercise time is 138 minutes, the exercise amount is 210 K-calories, the exercise quality is slow, and the number of steps is 12610. The configuration is such that this exercise amount is compared with the lifestyle factor information optimum value, and advice is selected at a prescribed timing by the advice selection unit, and is output from the advice output unit.

### Embodiment 3

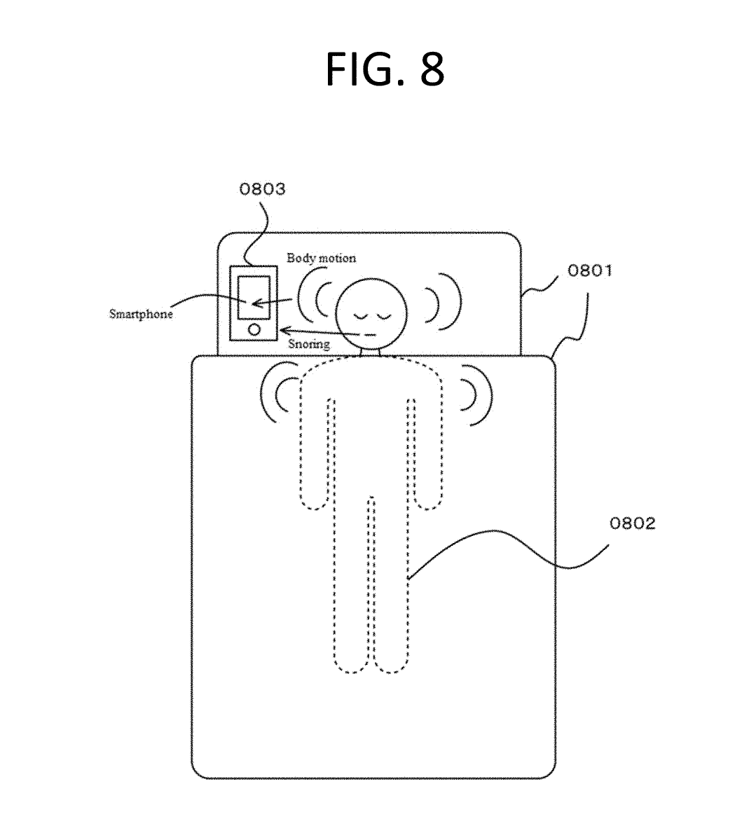

FIG. 8 shows an example of acquiring sleep data as lifestyle factor information. The cancer prevention/improvement advice device, which is a smartphone, is placed near the pillow of a user (0802), and uses a microphone as a lifestyle factor information acquisition unit (0803) to acquire data on the user while sleeping such as breathing sounds, snoring sounds, teeth grinding sounds, and voice sounds from talking in one's sleep, and acquires lifestyle factor information such as the sleep depth and number of hours from these types of information and an accumulated sleep database. Moreover, the microphone of this device gathers sounds caused by the body motion of the user such as rubbing sounds with the futon (0801), and rubbing sounds from the futon itself, and of course acquires the depth and length of sleep, etc. from the sleep database as lifestyle factor information. The obtained lifestyle factor information is sent to the lifestyle factor information accumulation unit, and is accumulated as history information.

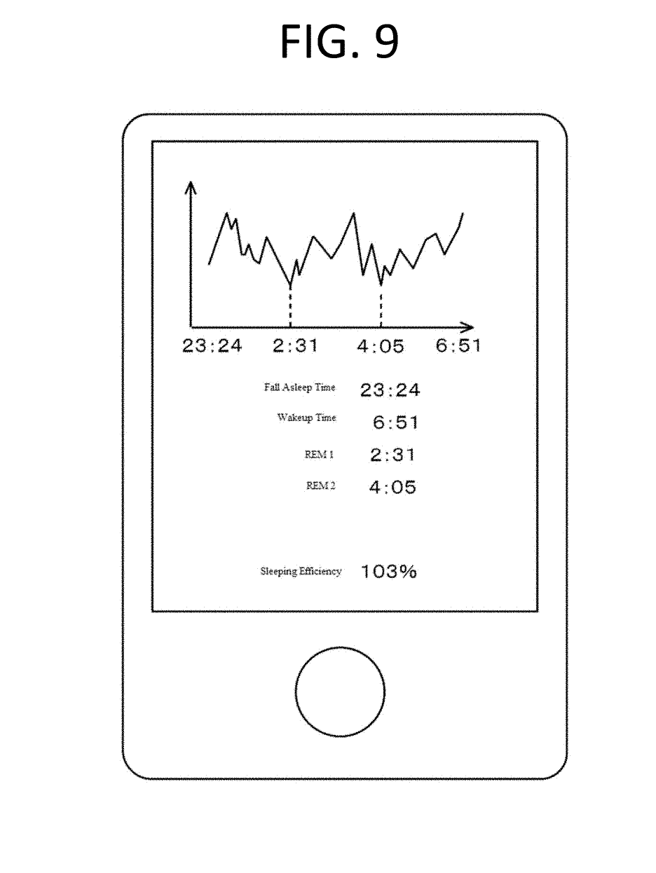

FIG. 9 is an example of lifestyle factor information displayed on a screen of a smartphone based on acquired and accumulated sleep data, which is a type of lifestyle factor information. Here, the depth and quality of sleep are shown on a time axis from the time the user went to bed until waking up, and the sleeping efficiency is displayed in comparison with past user data. These data items and the lifestyle factor information optimum values are compared, and the advice selection unit selects and outputs appropriate advice in accordance with the advice selection rules. For example, on the morning of a day in which it is determined that the user received a sufficient amount of sleep, advice such as “You got a good night's sleep. Today let's focus and try to get some exercise” is selected and output from the advice output unit.

### Embodiment 4

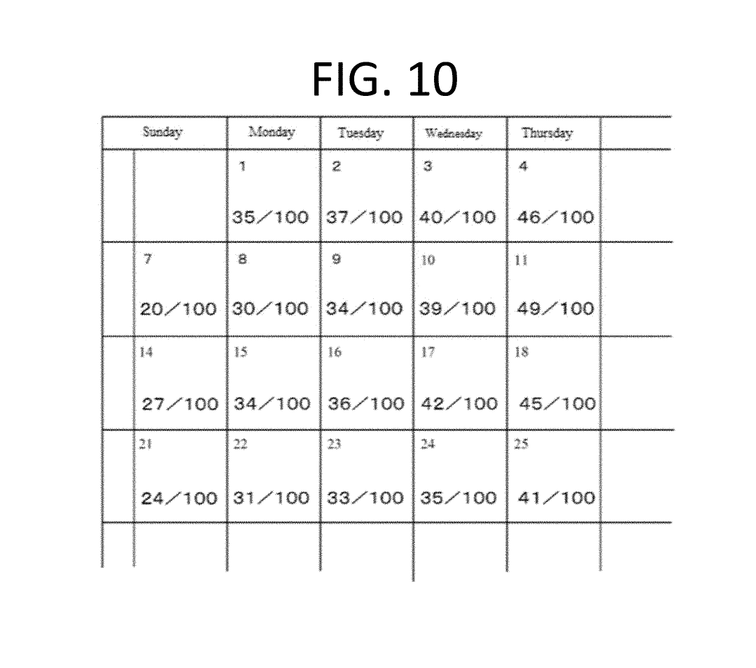

FIG. 10 illustrates an example in which stress data is acquired as lifestyle factor information. The reaction speed of the user's pupil to light is acquired from a video camera, which is a lifestyle factor information acquisition unit of a cancer prevention/improvement advice device, which is a smartphone, and stress data is acquired as lifestyle factor information.

FIG. 10 illustrates an example in which results obtained from evaluating stress as acquired lifestyle factor information are displayed in a calendar format. The calendar is prepared based on lifestyle factor information that is accumulated by the lifestyle factor information accumulation unit.

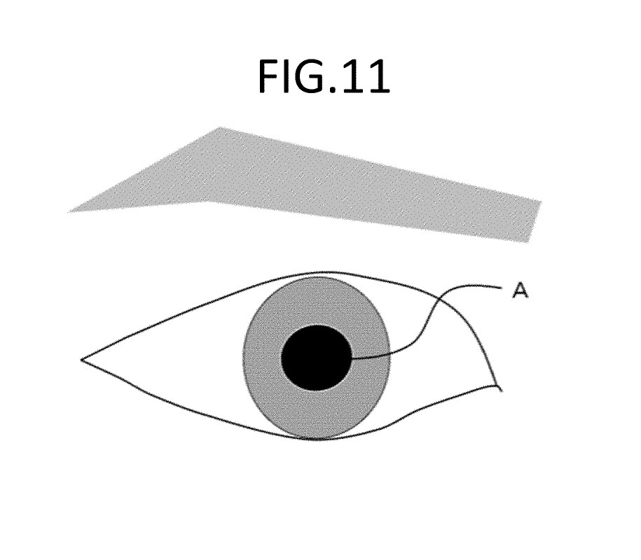

FIG. 11 is a schematic view of an eye for grasping pupillary changes. When a light is suddenly shined into the eye, the portion of the eye indicated by A in the figure becomes gradually smaller, but the speed at which the pupil becomes smaller is gradually slowed according to the amount of stress. In other words, when the user is under a great deal of stress, the conformity of the pupil to light worsens.

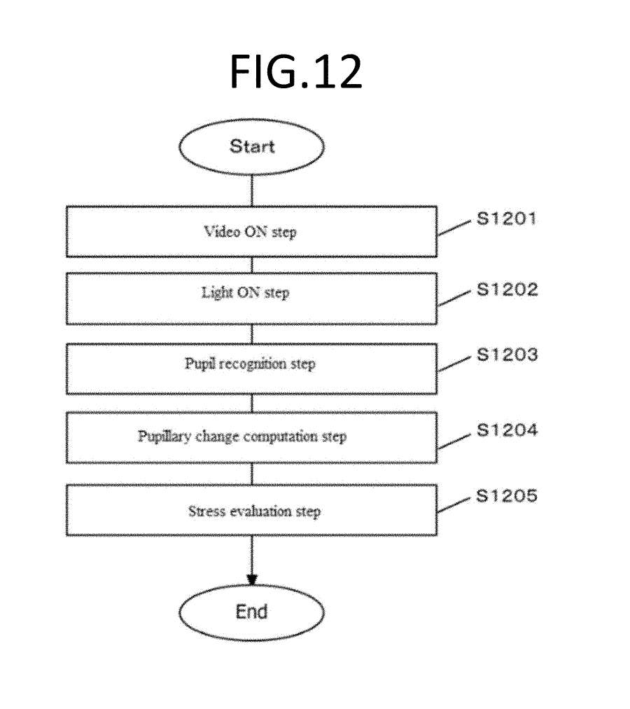

FIG. 12 shows the processing flow for obtaining with a cell phone the pupil's conformity to light, or in other words stress data, which is a type of lifestyle factor information. First, the filming of a video is started through the video ON step (S1201). At this time, the pupil must be filmed. A configuration for filming the pupil is conceivable in which, for example, image recognition technology is used to notify the user by sound or the like that the pupil is being filmed. Next is a light ON step (S1202). This is step in which the amount of light is increased to cause the pupil to constrict. The lighting function of a smartphone or the like can be used. Next, a pupil recognition step for measuring the pupil (S1203), and a pupillary change computation step (S1204) are performed, and the time from when a prescribed constriction of the pupil is observed until the pupil becomes stable, or the constriction time until a predetermined percentage of constriction, or the constriction time until a predetermined size is reached is measured, and converted to a stress value (stress data). These steps are performed by the lifestyle factor information acquisition unit of the cancer prevention/improvement advice device. Furthermore, the data is accumulated by the lifestyle factor information accumulation unit, output as shown by FIG. 10 for example, and used in the selection of advice. For example, for a case in which it has been determined on the basis of advice selection rules that the stress level during the most recent one week period was high, advice such as “How about slowly relaxing in a nice bath today” is selected and output from the advice output unit.

### Embodiment 5

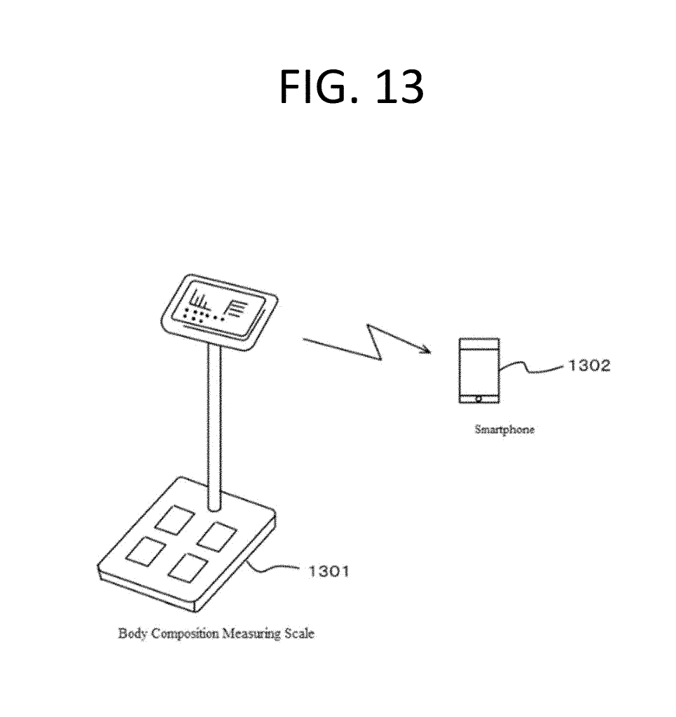

FIG. 13 is an image showing a specific example of acquiring body composition data as biological data. As shown in this image, a body composition measuring system (1301) measures various data such as the amount of intercellular moisture, amount of extracellular moisture, amount of protein, amount of minerals, amount of body fat, body weight, skeletal muscle mass, BMI, body fat percentage, waist-to-hip ratio, muscle balance (right arm, left arm, trunk, right leg, left left), ECW/TBW, obesity, BCM, BMC, BMR, AC, and AMC values, and these values are acquired through communication by the lifestyle factor information acquisition unit (1302), which is a cancer prevention/improvement advice device. The acquired lifestyle factor information is accumulated by the lifestyle factor information accumulation unit and compared with the lifestyle factor information optimum value, multiple pieces of data are synthesized, and an evaluation value is produced as lifestyle factor information. Advice is then selected in accordance with the advice selection rules on the basis of the extent of divergence from the registered lifestyle factor information optimum values. For example, if the amount of body fat suddenly increases compared to the previous day, advice such as “Let's get plenty of exercise today” is selected and output.

### Embodiment 6

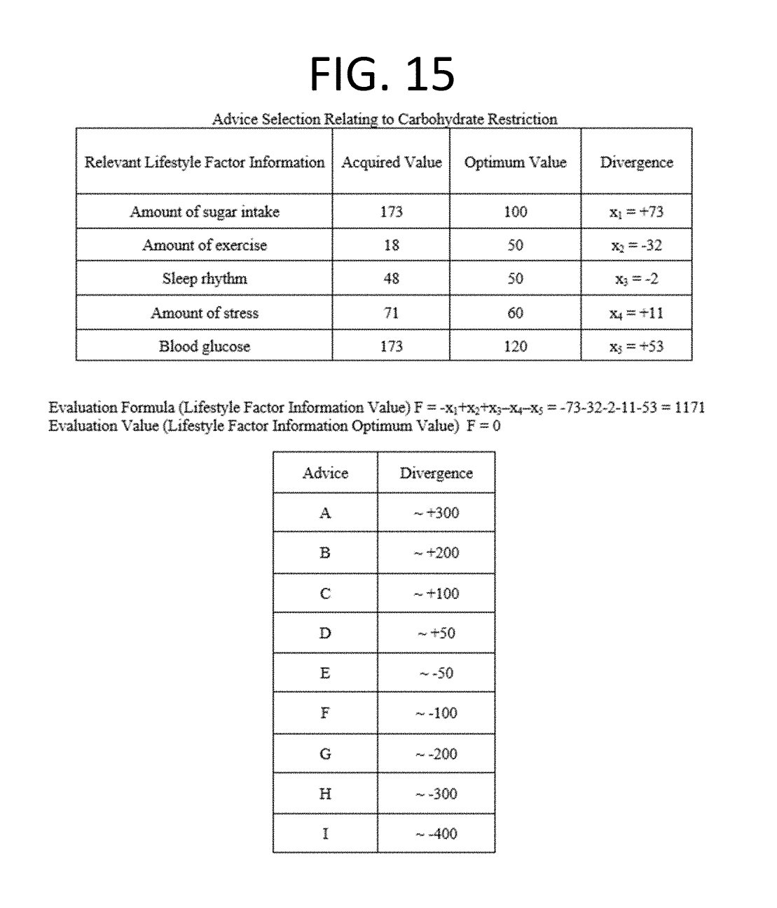

FIG. 15 is an image for explaining the mechanism for comprehensively using meal information, exercise data, sleep data, stress data, and biological data and selecting advice according to a purpose of restricting carbohydrates. For example, as shown in FIG. 15, for a case in which the optimum value of the amount of sugar intake is 100, the acquired value is 173, and the divergence is +73, the optimum value for the exercise amount is 50, the acquired value is 18, and the divergence is −32, the sleep rhythm (acquired amount of sleep or sleeping efficiency) optimum value is 50, the acquired value is 48, and the divergence is −2, the optimum value for the amount of stress (stress evaluation results) is 60, the acquired value is 71, and the divergence is +11, and the optimum value for blood glucose is 120, the acquired value is 173, and the divergence is +53, and the evaluation formula is an evaluation formula like that shown in FIG. 15, the lifestyle factor information value is −171 based on the evaluation formula and on the lifestyle factor information optimum value expressed by the evaluation formula being 0, and therefore an advice G is selected based on the advice selection rules shown at the bottom of the image.

In this manner, lifestyle factor information items of a plurality of categories can be comprehensively evaluated by an evaluation formula, and rules for selecting advice using these as lifestyle factor information items can be used.

### <System for Increasing Advice Precision Through Big Data Processing>

The following system is conceivable as a system for increasing advice precision through big data processing.

Namely, as an eighth aspect, the cancer prevention/improvement advice device according to claims 1 to 6 is proposed, wherein for a case in which the lifestyle factor information is acquired as a numeric value, the lifestyle factor information accumulation unit includes a first statistical processing means for determining, on the basis of past numeric values, an average value, a dispersion, a deviation value of data each time, a t-distribution, an F-distribution, and a chi-squared distribution.

Furthermore, as a ninth aspect, the cancer prevention/improvement advice device based on the eighth aspect is proposed, further having, for a case in which the lifestyle factor information is acquired as a numeric value, a lifestyle factor information cross-correlation coefficient acquisition unit for acquiring a lifestyle factor information cross-correlation coefficient, which is a cross-correlation coefficient for the lifestyle factor information accumulated by the lifestyle factor information accumulation unit.

Furthermore, as a tenth aspect, the cancer prevention/improvement advice device based on the eighth or ninth aspect is proposed, wherein the advice selection rule retaining unit has an advice first selection rule changing means for changing the retained advice selection rule on the basis of an average value, a dispersion, a deviation value of data each time, a t-distribution, an F-distribution, and a chi-squared distribution determined by the first statistical processing means of the lifestyle factor information accumulation unit.

Furthermore, as an eleventh aspect, the cancer prevention/improvement advice device according to the eighth or ninth aspect is proposed, wherein the lifestyle factor information accumulation unit further includes: an advice associated lifestyle factor information accumulation means for accumulating lifestyle factor information that is associated with advice output from the advice output unit and acquired; and an advice lifestyle factor information correlation coefficient acquisition unit for acquiring an advice lifestyle factor information correlation coefficient, which is a correlation coefficient between the associated advice and the lifestyle factor information.

Furthermore, as a twelfth aspect, the cancer prevention/improvement advice device according to the eleventh aspect is proposed, wherein the advice selection rule retaining unit includes a second advice selection rule changing means for changing the retained advice selection rule on the basis of the advice lifestyle factor information correlation coefficient acquired by the advice lifestyle factor information correlation coefficient acquisition unit.

As a thirteenth aspect, a cancer prevention/improvement advice system is proposed, the system including: the cancer prevention/improvement advice device according to any one of the first to sixth and the eighth to twelfth aspects further having: a history information output unit for outputting, through a network, the history information of the lifestyle factor information accumulated by the lifestyle factor information accumulation unit; a history information acquisition unit for acquiring, through a network, the history information output from the history information output unit; and a central server having: a central server correlation coefficient calculation unit for statistically processing the acquired history information, and calculating a lifestyle factor information cross-correlation coefficient, and a central server correlation coefficient output unit for outputting the calculated correlation coefficient.

Furthermore, as a fourteenth aspect, the cancer prevention/improvement advice system according to the thirteenth aspect is proposed, wherein the central server includes a second statistical processing unit for determining, on the basis of the acquired history information, an average value of each lifestyle factor information, a dispersion, a deviation value of data each time, a t-distribution, an F-distribution, and a chi-squared distribution.

Furthermore, as a fifteenth aspect, the cancer prevention/improvement advice system according to the thirteenth or fourteenth aspect is proposed, wherein the central server further includes: an advice selection command acquisition unit for acquiring an advice selection rule change command, which is a command for changing the advice selection rule retained by the advice selection rule retaining unit for each cancer prevention/improvement advice device on the basis of the correlation coefficient calculated by the central server correlation coefficient calculation unit; and an advice selection rule change command output unit for outputting the acquired advice selection rule change command to each cancer prevention/improvement advice device; and each cancer prevention/improvement advice device further includes: an advice selection rule change command acquisition unit for acquiring an advice selection rule change command output from an advice selection rule change command output unit; and an advice selection rule change unit for changing, on the basis of an acquired advice selection rule change command, an advice selection rule retained by the advice selection rule retaining unit.

### Embodiment 7

The cancer prevention/improvement advice device of the present embodiment acquires lifestyle factor information including the user's physiological activity and information indicating the degree of health from the user, associates the acquired lifestyle factor information with advice information corresponding to the acquisition period, accumulates the data as history information, and evaluates the lifestyle factor information using lifestyle factor information optimum values, which are optimum values for each lifestyle factor directed at the purpose of cancer prevention and improvement, to obtain evaluation results. The cancer prevention/improvement advice device of the present embodiment then learns the relationship between the evaluation results and the advice information corresponding to the evaluated lifestyle factor information, uses advice selection rules, which are rules for selecting advice from a plurality of advice for more closely approaching the optimum values, to select advice using history information of the lifestyle factor information and lifestyle factor information optimum values retained as lifestyle factors thereof as variables, and outputs the selected advice. The device may also be configured so as to record new advice.

Hereinafter, the functional configuration, hardware architecture, and processing flow are separately described.

### «Functional Configuration»

In the present embodiment, the cancer prevention/improvement advice device is configured from a lifestyle factor information acquisition unit (1601), a lifestyle factor information accumulation unit (1602), a lifestyle factor information evaluation unit (1603), an advice results learning unit (1604), a lifestyle factor information optimum value retaining unit (1605), an advice retaining unit (1606), an advice selection rule retaining unit (1607), an advice selection unit (1608), and an advice output unit (1609). Of these, the lifestyle factor information acquisition unit, the lifestyle factor information optimum value retaining unit, the advice retaining unit, and the advice output unit are as described above.

The lifestyle factor information accumulation unit functions to associate the acquired lifestyle factor information with the below-described advice information that is corresponded with the acquisition period of the lifestyle factor information thereof and output, and to accumulate the lifestyle factor information thereof as history information. Unlike the lifestyle factor information accumulation units of the abovementioned embodiments, with the present lifestyle factor information accumulation unit, the lifestyle factor information is “associated with the below-described advice information that is corresponded with the acquisition period of the lifestyle factor information thereof” and accumulated as history information. History information may span a long period of time such as the past five years, the past three years, or the past one year, may span an medium amount of time such as one month, two weeks, or one week, or may be a short period of time such as only the most recent one time. Through this, learning using artificial intelligence (AI) can be performed.

The lifestyle factor information evaluation unit functions to evaluate lifestyle factor information using the below-described lifestyle factor information optimum values and to thereby obtain evaluation results. The evaluation results that are obtained here are used in learning the relationship with advice information.

The advice results learning unit functions to learn the relationship between the evaluation results and the advice information that corresponds to the evaluated lifestyle factor information. For example, the correlation relationship is analyzed from the advice details and the change in the accumulated lifestyle factor information from the perspective of efficiency for approaching the lifestyle factor information optimum values. Furthermore, from the analysis results, advice with a large effect is learned.

The advice selection rule retaining unit functions to retain advice selection rules, which are rules for selecting retained advice using history information of lifestyle factor information, lifestyle factor information optimum values retained for those lifestyle factors, and the learning results. Here, advice selection rules are changed based on the details learned by the advice results learning unit. Through this, the precision of advice can be improved.

The advice selection unit functions to select advice using the retained advice selection rules with the history information of lifestyle factor information and the lifestyle factor information optimum values retained for those lifestyle factors being used as variable.

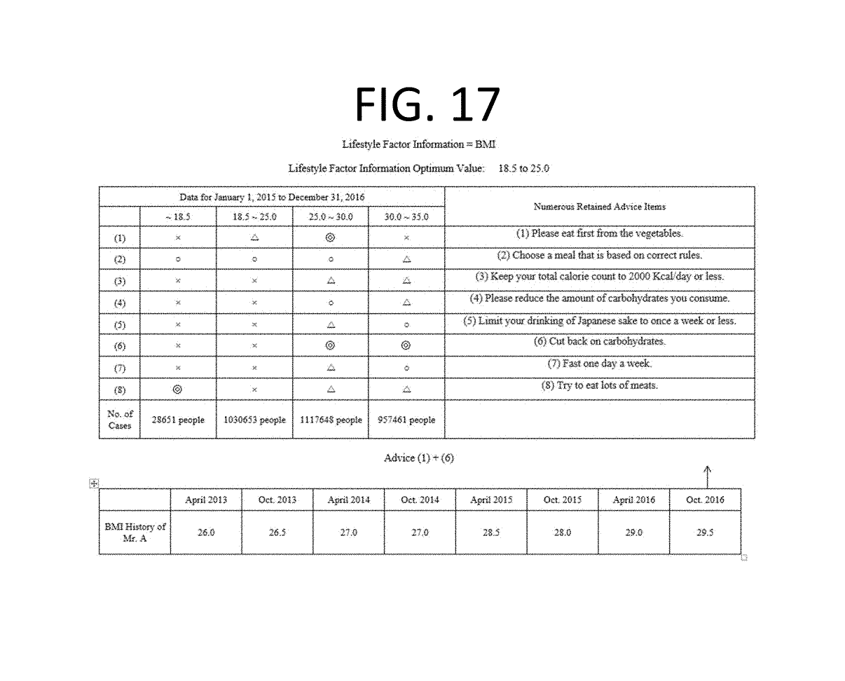

FIG. 17 is an image showing specific processing for selecting advice. As one example, the lifestyle factor information is described as a BMI value. The lifestyle factor information optimum value for BMI is 18.5 to 25.0. As retained advice, examples of a plurality of advice information items include “(1) Please eat first from the vegetables,” “(2) Choose a meal that is based on correct rules,” (3) Keep your total calorie count to 2000 Kcal/day or less,” “(4) Please reduce the amount of carbohydrates you consume,” “(5) Limit your drinking of Japanese sake to once a week or less,” “(6) Cut back on carbohydrates,” “(7) Fast one day a week,” and “(8) Try to eat lots of meats.” The table of FIG. 17 shows that of the advice given to 28651 people having a BMI value of less than 18.5 in a period from Jan. 1, 2015 to Dec. 31, 2016, the advice of “(2) Choose a meal that is based on correct rules” was effective, the advice of “(8) Try to eat lots of meats” was extremely effective, and the other advice items did not have an effect. Next, of the advice given to 1030653 people having a BMI value from 18.5 to less than 25.0, FIG. 17 shows that the advice of “(2) Choose a meal that is based on correct rules” was effective, the advice of “(1) Please eat first from the vegetables” was somewhat effective, and the other advice items did not have an effect. Note that in the case of a BMI value of from 18.5 to less than 25.0, because this range is originally the range of optimum values for the BMI value, the effect referred to here is an effect of maintaining the present condition. Next, of the advice given to 1117648 people having a BMI value from 25.0 to less than 30.0, FIG. 17 shows that the advice items of “(1) Please east first from the vegetables” and “(6) Cut back on carbohydrates” were extremely effective, the advice items of “(2) Choose a meal that is based on correct rules” and “(4) Please reduce the amount of carbohydrates that you consume” were effective, and the other advice items were slightly effective. Next, of the advice given to 957461 people having a BMI value from 30.0 to less than 35.0, FIG. 17 shows that the advice item of “(6) Cut back on carbohydrates” was extremely effective, the advice items of “(5) Limit your drinking of Japanese sake to once a week or less” and “(7) Fast one day a week” were effective, the advice items of “(2) Choose a meal that is based on correct rules,” “(3) Keep your total calorie count to 2000 Kcal/day or less,” “(4) Please reduce the amount of carbohydrates you consume,” and “(8) Try to eat lots of meats” were slightly effective, and the advice of “(1) Please east first from the vegetables” had no effect. From these data, the appropriate advice for users of each numeric value range of BMI values is analyzed and learned, and on the basis thereof, advice selection rules are updated, and of course, effective advice is suitable as appropriate advice details. For example, if we use Mr. A from the table of FIG. 17 as an example, his BMI in October 2016 was 29.5, and therefore the advice that would be selected as advice for Mr. A would be the advice “(1) Please east first from the vegetables” and “(6) Cut back on carbohydrates”, which are advice items that were learned to be extremely effective for the given numeric value.

In addition to the abovementioned functions, the cancer prevention/improvement advice device of the present invention may be further provided with a new advice recording unit for recording new advice in the advice retaining unit. When this is done, for cases in which is it thought that advice other than existing advice is appropriate from the learned results, appropriate advice can be automatically recorded. When this is done, more optimum advice is included in the advice that is selected by the advice selection unit, and the precision of the advice that is output can be further improved.

The method for measuring and the method for inputting lifestyle factor information are not particularly limited, and for example, the configuration may be such that data is obtained by a measurements device that is external from the device of the present invention, and that data is received by the device of the present invention. Furthermore, for example, a configuration may be adopted so that substances such as supplements and drugs that are taken can be identified using camera recognition.

### «Lifestyle Factor Information»

The lifestyle factor information that is acquired as body diagnosis results information by the lifestyle factor information acquisition unit may include any one or more of body measurement results (BMI, abdominal girth, visceral fat, body fat percentage), body status examination results (blood pressure, visual acuity, ocular pressure, pulmonary function, hearing capability, hair conditions), urinalysis results, stool examination results, number of times of stool passage, stool color, stool characteristics, blood biochemical test results, serological examination results, tumor marker examination results, diabetes mellitus examination results, biopsy examination results, fundus camera examination results, breast examination results, ultrasound examination results, gastrointestinal tract examination results, chest X-ray examination results, CT examination results, MRI examination results, PET examination results, rectal examination results, rectal temperature, electrocardiogram examination results, equilibrium function examination results, arteriosclerosis evaluation results, osteoporosis examination results, female hormone examination results, risk marker examination results, and genetic information. More specifically, urinalysis results may include the presence or lack of protein in the urine, sugar in the urine, urine occult blood reactions, bilirubin in the urine, and urobilinogen, etc., the urine pH value, and urinary sediment values. The configuration may be such that a measurement device is provided on a toilet, and these data items are measured when the user uses the bathroom. In addition, more specific stool examination results include the presence or lack of occult blood reactions, and the presence or lack of parasites. Also, regarding the number of times of stool passage, the stool color, and stool characteristics, a method of acquiring images photographed with a camera device may be used. In addition, blood biochemical test results may include the amounts and concentrations of total protein, albumin, cholinesterase, lactate dehydrogenase, aspartate transaminase, alanine transaminase, gamma-glutamyltransferase, alkaline phosphatase, total bilirubin, amylase, urea nitrogen, creatinine, creatine kinase, total cholesterol, neutral fats, HDL cholesterol, LDL cholesterol, uric acid, sodium, potassium, chlorine, calcium, inorganic phosphorous, serum iron, unsaturated iron binding capacity, and the like. Examples of serological examination results include the presence and amounts of antibodies resistant to bacteria and foreign substances, allergen details, and immune abnormalities. Examples of tumor marker examination results include values such as CA125, CA19-9, CEA, α-fetoprotein (AFP), ferritin, prostate-specific antigen (PSA), and TPA. Results relating to diabetes mellitus examinations include fasting blood glucose, blood glucose 2 hours after a glucose load, blood glucose at any time, and the HbA1c value. Examples of biopsy examination results include whether results belong to classifications such as NILM, ASC-US, ASC-H, LSIL, HSIL, and SCC. Examples of fundus camera examination results include details and the presence or lack of abnormal findings such as glaucomatous cupping magnification, tigroid ocular fundus, hard white exudate, drusen, suspicions of glaucoma, bleeding, glaucoma, chorioretinal atrophy, suspicions of cataracts, optic chiasm phenomenon, cataracts, suspicions of bleeding, white exudate, retinal degeneration, meandering, epiretinal membrane, suspicions of macular degeneration, macular findings, conus, and papilla part findings. Breast examination results include findings such as calcification, tumors, and localized asymmetric shadows. Examples of breast ultrasound examination results include breast cysts, breast fibroadenomas, calcification, tumors, ductal ectasia, mastitis, and mastopathy. Examples of gastrointestinal tract examination results include the presence or lack of findings such as achalasia, esophageal varices, hiatal hernias, gastroptosis and a cascade stomach, atrophic gastritis, erosive gastritis, elevated lesions and translucency, depressed lesions, mucosal irregularities, mucosal folds, edge irregularities, retractions, diverticulum, ulcerative scars, the presence or lack of Helicobacter pylori antibodies, and the concentration of pepsinogen. Examples of chest X-ray examination results include the presence or lack of abnormal shadows. Examples of CT examination results, MRI examination results, PET examination results, rectal examination results, electrocardiogram examination results, arteriosclerosis evaluation results, and risk marker examination results includes the presence or lack of abnormal findings and the details thereof. Examples of equilibrium function examination results include the presence or lack of swaying appearing in the body when the eyes are opened and when the eyes are closed, and if present, the extent of swaying, the tendency for falling, and observed results such as differences when eyes are opened and closed. Examples of osteoporosis examination results include bone density, microstructure of the bone, the rate of bone metabolism turnover, the presence or lack of micro-fractures, and the density of calcifications. Examples of female hormone examination results include estradiol values, LH and FSH gonadotropin hormone values, and prolactin levels.

Next, lifestyle factor information acquired by the lifestyle factor information may include any one or more of the following daily life information such as the light quality and brightness of the living environment, amount of noise in the living environment, room temperature of the living environment, humidity of the living environment, light quality and brightness of the workplace environment, amount of noise in the workplace environment, room temperature of the workplace environment, humidity of the workplace environment, meal content, calorie intake from meals, food product ingredients and amount ingested with meals, number of meals, timing of meals, details of ingested supplements, intake calories from ingested supplements, ingested supplement ingredients and amounts from ingested supplements, number of times of ingestion of ingested supplements, ingestion timing of ingested supplements, quality of sleep, amount of sleep, type of exercise and amount of exercise, quality of work and amount of work, working hours, amount of movement at work, commute quality and amount, evaluation results from company at work (salary increase, job promotion, job transfer, change in assignment), presence or lack of unemployment and career change, quality and amount of training to maintain and increase physical strength, quality and amount of walking around town, quality and amount of shopping, quality and amount of contact with the natural environment, quality and amount of entertainment, quality and amount of friendships, quality and amount of marital relations, type and amount of accidents, presence or lack of residence changes, presence or lack of marriage and divorce, purchase history of expensive articles and expensive goods, quality and amount of learning for obtaining qualifications, presence or lack of qualification acquisition, and change in relationship with customers for cases of company management and self-employment. More specifically, examples of light quality and brightness include values such as luminous flux [lm: lumens], luminous intensity [cd: candela], brightness [cd/m²: candela/square meter], and illuminance [lx:lux]. Examples relating to noise amounts include the number of pascals (Pa), which shows the physical amount that indicates the magnitude (pressure) of noise, and the number of decibels (dB), which indicates the sound pressure level. Examples of meal content data include information by food type such as convenience store food, retort-packaged food, home cooked meals, and food from a family restaurant. Examples of food product ingredients and amounts ingested with meals include energy, water content, proteins, proteins from amino acid compositions, fats, triacylglycerol equivalents, saturated fatty acids, monounsaturated fatty acids, polyunsaturated fatty acids, cholesterol, carbohydrates, water soluble dietary fiber, insoluble dietary fiber, ash, sodium, potassium, calcium, magnesium, phosphorous, iron, zinc, copper, manganese, iodine, selenium, chromium, molybdenum, vitamin A (retinol, α-carotene, β-carotene, β-cryptoxanthin, β-carotene equivalents, retinol activity equivalents), vitamin D, vitamin E (α-tocopherol, β-tocopherol, γ-tocopherol, δ-tocopherol), vitamin K, vitamin B₁, vitamin B₂, niacin vitamins, vitamin B₆, vitamin B₁₂, folic acid vitamins, pantothenic acid vitamins, biotin vitamin, vitamin C, sodium chloride equivalents, and alcohol. Details of ingested supplements include amounts of vitamin A, vitamin B, vitamin C, vitamin D, vitamin E, vitamin H, vitamin K, vitamin P, vitamin U, cobalamin, zinc, iron, copper, chromium, selenium, magnesium, calcium, potassium, sodium, cobalt, molybdenum, iodine, phosphorous, MSM, tryptophan, threonine, leucine, isoleucine, lysine, methionine, phenylalanine, histidine, asparagine, serine, proline, glutamine, tyrosine, DNA, α-linolenic acid, EPA, DHA, arachidonic acid, gamma-linolenic acid, DNJ, vegetable juice made from green leafy vegetables, egg yolk choline, Grifola frondosa, ephedra, Monascus purpureus, germinated brown rice, lactic acid bacteria, natto, nano bacteria, Panax notoginseng, Korean ginseng, evening primrose, plant sterols, perilla oil, linseed oil, Asian ginseng, mulberry leaf, plum extract, liver oil extract, red wine extract, soy isoflavones, Ganoderma lucidim, green tea, green barley, dietary fiber, black beans, Agaricus, acerola, Arabino xylan, allicin, alkoxyglycol, aloe, Andes carrots, isoflavones, Ginkgo biloba leaves, India carrots, turmeric, spring turmeric, fall turmeric, echinacea, Hypericum perforatum (ST. John's-wort), Siberian ginseng, Nico-n, Enokitake, ephedra, euginin, O-brief, oligosaccharides, Kayapo potatoes, catechins, gaukurua, guarana, garcinia, calcium, chitin, chitosan, cat's claw, cabbage, Gymnema, ginkoba, quercetin, citric acid, cranberries, curcumin, chlorella, glucosamine, kale, kefir, coenzyme Q10, sesame, collagen, chondroitin, chondroitin sulfate, goya, shark cartilage, Siberian ginseng, squalene, ginsenosides, silymarin, zeoxanthin, saw palmetto, sweet clover, turmeric, thiamine, chaste tree, theanine, tocotrienol, tocopherol, tomato, docosahexaenoic acid, thickening agents, nano kinase, bitter melon, Nico-n, nicotinic acid, nicotinamide, garlic, saw palmetto, Noni, BioBran, banaba, rose flower extract, valerian, papaya, papain, pantothenic acid, hyaluronic acid, brewer's yeast, pyconogenol, Pikopen, Lactobacillus bifidus, Agaricus blazei Murrill, pyridoxine, pyridoxal phosphate, pyridoxamine, bilberry, pink grapefruit, phylloquinone, fucoidan, black cohosh, blueberries, pueraria mirifica, phosphatidylcholine, pteroylglutamic acid, proanthocyanidins, propolis, beta-glucan, borage oil, maca, milk thistle, mumefural, Phellinus methylsulfonylmethane, menaquinone, melatonin, melilot, monacolin, lycopene, riboflavin, lutein, lychee, lecithin, retinal, retinoic acid, retinol, red clover, royal jelly, and rose hip. Sleep quality includes details such as the balance between REM sleep, which is deep sleep, and non-REM sleep. The amount of sleep includes details such as the number of hours of sleep. Details on the types of exercise include details by aerobic exercise and anaerobic exercise, stretching, muscle training and other such specific exercise details. Details on the amount of exercise include, for example, the amount of exercise time and the number of calories that were consumed. Details on the quality of work are provided by job details such as whether a job is a clerical position or a sales position, and by managerial position with regard to the importance of roles and responsibilities. The amount of work includes details such as the number of hours of work and the amount of results that are obtained. The amount of movement at work includes details such as the movement distance and the time required for movement. The commute quality includes details by the commuting means, and for example, if commuting by train, the extent of crowding, and the like. As the quality and amount of training to maintain and increase physical strength, information may include the training details and the amount of training time and frequency. Details on the quality an amount of walking around town include, for example, the location and speed of walking around town, and the time and frequency. Examples of details on the quality and amount of shopping include the shopping location, details of purchased goods, and the amount and cost of purchase goods. Details regarding the quality and amount of contact with the natural environment include details of contact with plants and animals, and frequency and time. Details regarding the quality and amount of entertainment include, for example, entertainment details, win/loss details if the entertainment involves winning or losing, and the entertainment frequency, expense, and time spent. Details on the quality and amount of friendships, and marital relations include, for example, the frequency of communication and contact, contact details, conversation details, and contact time. Details on the types and amounts of accidents include, for example, accident details, the presence or lack of injuries due to accidents and details of the injuries thereof, and details categorized by the accident perpetrator and victim such as details of the response of the other party, and the frequency of accidents. Details on the quality and amount of learning for obtaining qualifications include, for example, the level of difficulty of qualification testing, the level of impact that qualification acquisition has on life, the presence or lack of goal achievement, and the amount of learning.

Next, the lifestyle factor information acquired as illness and injury information by the lifestyle factor information acquisition unit may be any one or more types of information such as the types and amounts of injuries and illnesses, the types and amounts of drugs that are taken according to the injury and illness, the frequency and time length of hospital visits according to injuries and illnesses, the types and amounts of diagnoses performed in accordance with injuries and illnesses, the types and amounts of surgeries performed in accordance with injuries and illnesses, the devices carried on the body in accordance with injuries and illnesses, tools for assistance, and the types and amounts of bandages.

Next, the lifestyle factor information acquired as mental status information by the lifestyle factor information acquisition unit may be any one or more types of information such as the voice quality of the user, the voice magnitude of the user, an amount of speech by the user, the status of autonomic nerves, the user's mood, the level of satisfaction with sex life, embraced image of an item, mood when getting out of bed, mood when falling asleep, mood during sleep, mood during work, mood on holidays, mood during meals, mood when defecating, effective body temperature, coldness of hands and feet, and ease of respiration. More specifically, details of the voice quality of the user can include, for example, the highness and tension of the voice, and the nasal sounding of the voice. It is conceivable that the voice magnitude of the user can be measured with a unit of decibels (db). Details of the status of autonomic nerves can include, for example, details with regard to sympathetic nerve dominance and parasympathetic nerve dominance. Examples of the user's mood can include pleasant, unpleasant, positive, negative, and the like. The level of satisfaction with the sex life may be numerically quantified, or may be ranked at levels such as “unsatisfied”, “normal”, “satisfied” and “extremely satisfied”. Details of embraced images of items can include, for example, emotions such as joy, anger, grief, comfort, love, hatred, evil, and greed. With regard to the mood when getting out of bed, mood when falling asleep, mood during sleep, mood during work, mood on holidays, mood during meals, and mood when defecating, numeric values may be assigned to feelings such as comfort and discomfort. As the coldness of hands and feet, the temperature at the tips of the hands and feet may be measured through a thermography examination or the like.

### «Genetic Information»

Genetic information may also be acquired in addition to the above lifestyle factor information. Depending on the genes, specific tendencies such as the ease of developing cancer may exist. Therefore, even in the selection of advice, selection that takes into account this genetic information is desirable, and with regard to the retained advice selection rules, it is desirable that rules be applied using also genetic information as a variable in addition to the history information of lifestyle factor information and the lifestyle factor information optimum values retained for the lifestyle factors.

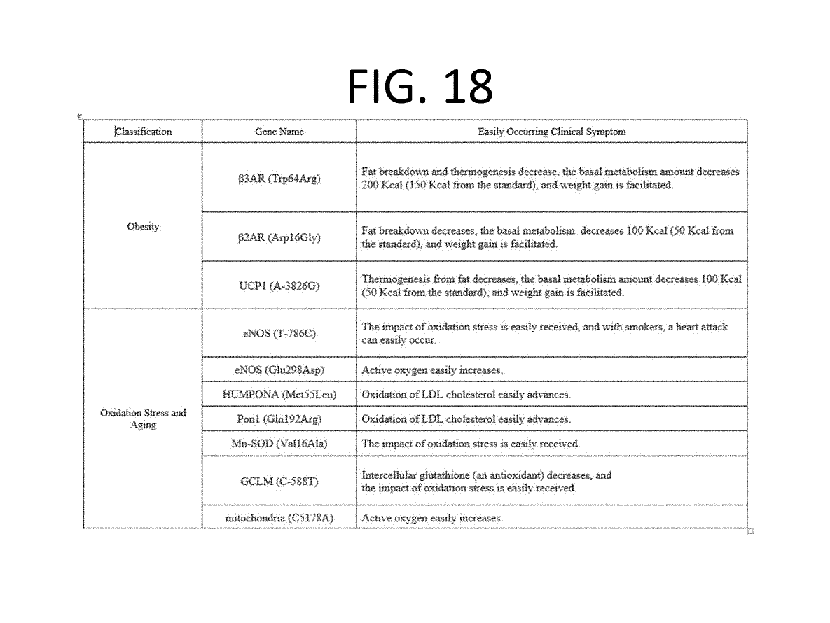

FIG. 18 is an image illustrating an example of genetic information. For example, when described with respect to the classification of “obesity”, when the “β3AR (Trp64Arg)” gene is present, a clinical symptom of “fat breakdown and thermogenesis decrease, the basal metabolism amount decreases 200 Kcal (150 Kcal from the standard), and weight gain is facilitated” can easily occur, and when the “β2AR (Arp16Gly)” gene is present, a clinical symptom of “fat breakdown decreases, the basal metabolism decreases 100 Kcal (50 Kcal from the standard), and weight gain is facilitated” can easily occur, and when the “UCP1 (A-3826G)” gene is present, a clinical symptom of “thermogenesis from fat decreases, the basal metabolism amount decreases 100 Kcal (50 Kcal from the standard), and weight gain is facilitated” easily occurs. Furthermore, when described with respect to the classification of “oxidation stress and aging”, when the “eNOS (T-786C)” gene is present, the clinical symptom of “the impact of oxidation stress is easily received, and with smokers, a heart attack can easily occur” is facilitated, when the “eNOS (Glu298Asp)” gene is present, the clinical symptom of “active oxygen easily increases” easily occurs, when the “HUMPONA (Met55Leu)” gene is present, the clinical symptom of “oxidation of LDL cholesterol easily advances” easily occurs, when the “Pon1 (Gln192Arg)” gene is present, the clinical symptom of “oxidation of LDL cholesterol easily advances” easily occurs, when the “Mn-SOD (Val16Ala)” gene is present, the clinical symptom of “the impact of oxidation stress is easily received” easily occurs, when the “GCLM (C-588T)” gene is present, the clinical symptom of “intercellular glutathione (an antioxidant) decreases, and the impact of oxidation stress is easily received” easily occurs, and when the “mitochondria (C5178A)” gene is present, the clinical symptom of “active oxygen easily increases” easily occurs.

### «Lifestyle Factor Information Optimum Values»

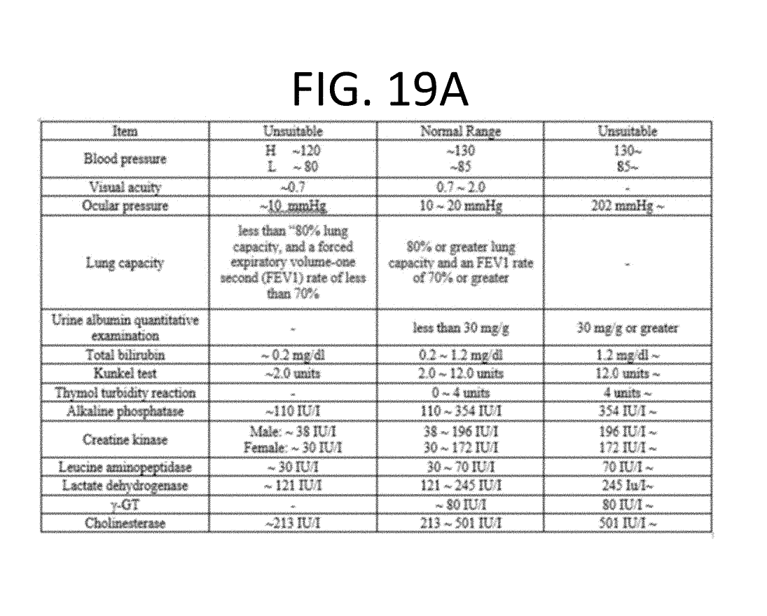

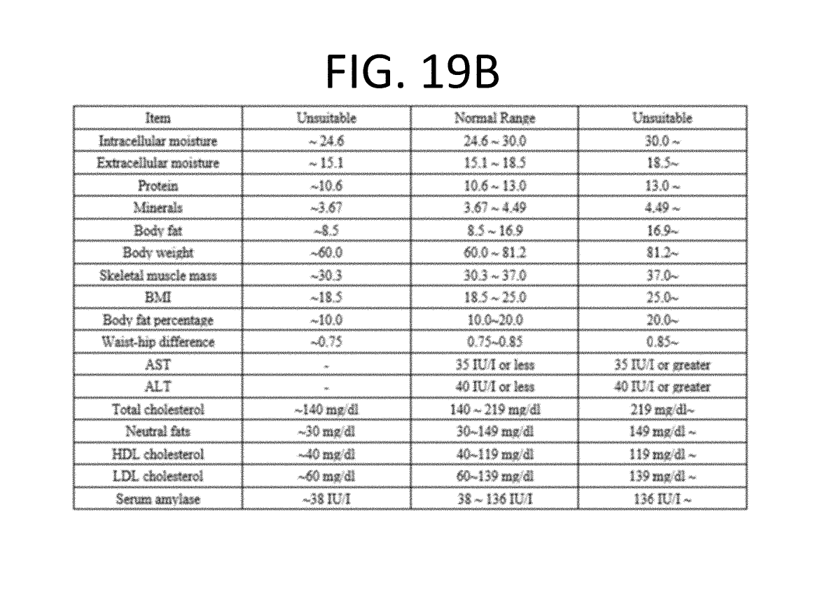

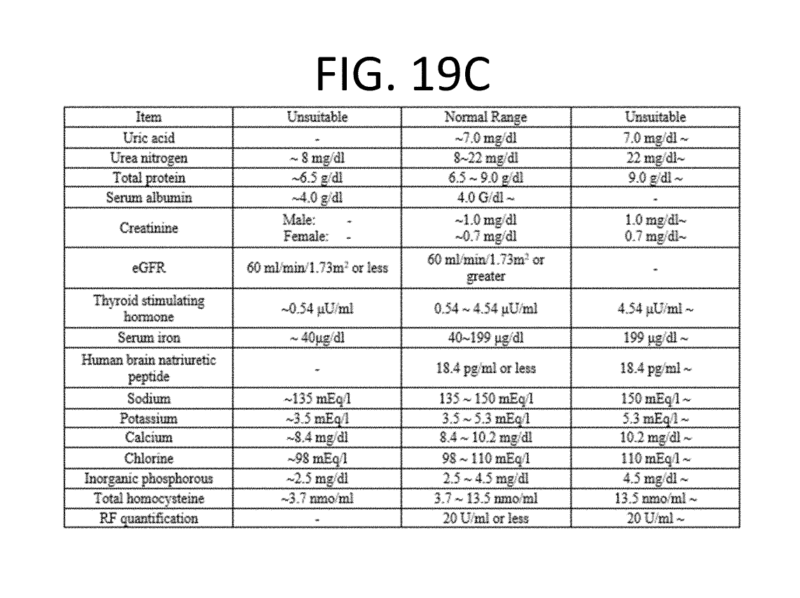

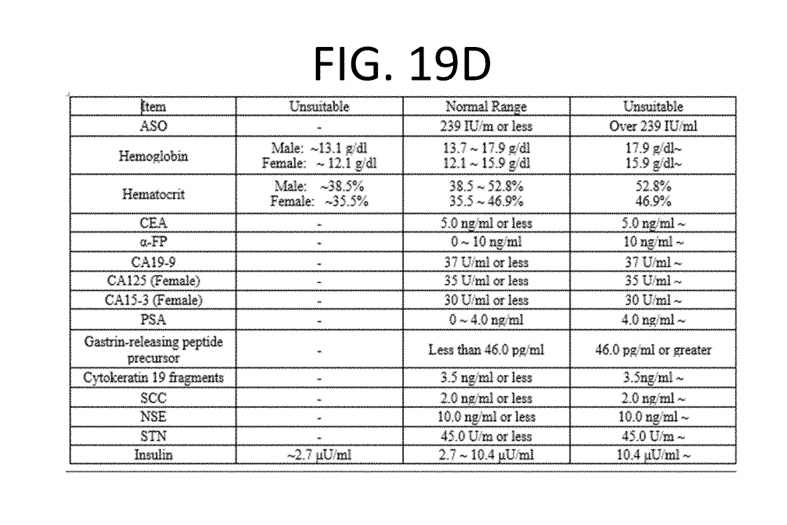

FIG. 19A to FIG. 19D are tables showing a specific example of lifestyle factor information optimum values. Regarding blood pressure, a high value of “less than 120” is unsuitable, a range of “120 to 130” is the normal range, and a value “over 130” is unsuitable; a low value of “less than 80” is unsuitable, a range of “80 to 85” is the normal range, and “over 85” is unsuitable. Regarding visual acuity, a value of “less than “0.7” is unsuitable, and a range of “0.7 to 2.0” is the normal range. Regarding ocular pressure, a value of “less than 10 mmHg” is unsuitable, a range of “10 to 20 mmHg” is the normal range, and “over 20 mmHg” is unsuitable. Regarding lung capacity, a value of “less than “80% lung capacity, and a forced expiratory volume-one second (FEV1) rate of less than 70%” is unsuitable, and “80% or greater lung capacity and an FEV1 rate of 70% or greater” is the normal range. Regarding urine albumin, the normal range is “less than 30 mg/g”, and a value of “30 mg/g or greater” is unsuitable. Regarding the total bilirubin, a value of “less than 0.2 mg/dl” is unsuitable, a range of “0.2 to 1.2 mg/dl” is the normal range, and values that “exceed 1.2 mg/dl” are unsuitable. Regarding values from a Kunkel test, values of “less than 2.0” are unsuitable, a range of “2.0 to 12.0” is the normal range, and values of “over 12.0” are unsuitable. Regarding a thymol turbidity reaction, values of “0 to 4” are in a normal range, and “over 4” is unsuitable. Regarding alkaline phosphatase, “less than 110 IU/I” is unsuitable, a range of “110 to 354 IU/I” is the normal range, and “over 354 IU/I” is unsuitable. Regarding creatine kinase, in the case males, a value of “less than 38 IU/I” is unsuitable, a range of “38 to 196 IU/I” is the normal range, and “over 196 IU/I” is unsuitable, and in the case of females, a value of “less than 30 IU/I” is unsuitable, “30 to 172 IU/I” is the normal range, and “over 172 IU/I” is unsuitable. Regarding leucine aminopeptidase, a value of “less than 30 IU/I” is unsuitable, “30 to 70 IU/I” is the normal range, and “over 70 IU/I” is unsuitable. Regarding lactate dehydrogenase, a value of “less than 121 IU/I” is unsuitable, “121 to 245 IU/I” is the normal range, and “over 245 IU/I” is unsuitable. Regarding γ-GT, a value of “less than 80 IU/I” is in the normal range, and “over 80 IU/I” is unsuitable. Regarding cholinesterase, a value of “less than 213 IU/I” is unsuitable, “213 to 501 IU/I” is the normal range, and “over 501 IU/I” is unsuitable. Regarding the amount of intercellular moisture, “less than 24.6” is unsuitable, “24.6 to 30.0” is the normal range, and “over 30.0” is unsuitable. Regarding the amount of extracellular moisture, “less than 15.1” is unsuitable, “15.1 to 18.5” is the normal range, and “over 18.5” is unsuitable. Regarding the amount of protein, “less than 10.6” is unsuitable, “10.6 to 13.0” is the normal range, and “over 13.0” is unsuitable. Regarding the amount of minerals, “less than 3.67” is unsuitable, “3.67 to 4.49” is the normal range, and “over 4.49” is unsuitable. Regarding the amount of body fat, “less than 8.5” is unsuitable, “8.5 to 16.9” is the normal range, and “over 16.9” is unsuitable. Regarding body weight, “less than 60.0” is unsuitable, “60.0 to 81.2” is the normal range, and “over 81.2” is unsuitable. Regarding skeletal muscle mass, “less than 30.3” is unsuitable, “30.3 to 37.0” is the normal range, and “over 37.0” is unsuitable. Regarding BMI, “less than 18.5” is unsuitable, “18.5 to 25.0” is the normal range, and “over 25.0” is unsuitable. Regarding the body fat percentage, “less than 10.0” is unsuitable, “10.0 to 20.0” is the normal range, and “over 20.0” is unsuitable. Regarding the waist-hip difference, “less than 0.75” is unsuitable, “0.75 to 0.85” is the normal range, “over 0.85” is unsuitable. Regarding AST, “35 IU/I or less” is the normal range, and “over 35 IU/I” is unsuitable. Regarding ALT, “40 IU/I” is the normal range, and “over 40 IU/I” is unsuitable. Regarding total cholesterol, “less than 140 mg/dl” is unsuitable, “140 to 219 mg/dl” is the normal range, and “over 219 mg/dl” is unsuitable. Regarding neutral fats, “less than 30 mg/dl” is unsuitable, “30 to 149 mg/dl” is the normal range, and “over 149 mg/dl” is unsuitable. Regarding HDL cholesterol, “less than 40 mg/dl” is unsuitable, “40 to 119 mg/dl” is the normal range, and “over 119 mg/dl” is unsuitable. Regarding LDL cholesterol, “less than 60 mg/dl” is unsuitable, “60 to 139 mg/dl” is the normal range, and “over 139 mg/dl” is unsuitable. Regarding serum amylase, “less than 38 IU/I” is unsuitable, “38 IU/I to 136 IU/I” is the normal range, and “over 136 IU/I” is unsuitable. Regarding uric acid, “less than 0.7 mg/dl” is the normal range, and “over 0.7 mg/dl” is unsuitable. Regarding urea nitrogen, “less than 8 mg/dl” is unsuitable, “8 to 22 mg/dl” is the normal range, and “over 22 mg/dl” is unsuitable. Regarding total protein, “less than 6.5 g/dl” is unsuitable, “6.5 to 9.0 g/dl” is the normal range, and “over 9.0 g/dl” is unsuitable. Regarding serum albumin, “less than 4.0 g/dl” is unsuitable, and “4.0 g/dl or greater” is the normal range. Regarding creatinine, in the case of males, “less than 1.0 mg/dl” is the normal range, and “1.0 mg/dl or higher” is unsuitable, and in the case of females, “less than 0.7 mg/dl” is the normal range, and “0.7 mg/dl or higher” is unsuitable. Regarding eGFR, “less than 60 ml/min/1.73 m²” is unsuitable, and “60 ml/min/1.73 m² or greater” is the normal range. Regarding the thyroid stimulating hormone, “less than 0.54 μU/ml” is unsuitable, “0.54 to 4.54 μU/ml” is the normal range, and “over 4.54 μU/ml” is unsuitable. Regarding serum iron, “less than 40 μg/dl” is unsuitable, “40 to 199 μg/dl” is the normal range, and “over 199 μg/dl” is unsuitable. Regarding human brain natriuretic peptide, “18.4 pg/ml or less” is the normal range, and “over 18.4 pg/ml” is unsuitable. Regarding sodium, “less than 135 mEq/l” is unsuitable, “135 to 150 mEq/l” is the normal range, and “over 150 mEq/l” is unsuitable. Regarding potassium, “less than 3.5 mEq/l” is unsuitable, “3.5 to 5.3 mEq/l” is the normal range, and “over 5.3 mEq/l” is unsuitable. Regarding calcium, “less than 8.4 mg/dl” is unsuitable, “8.4 to 10.2 mg/dl” is the normal range, and “over 10.2 mg/dl” is unsuitable. Regarding chlorine, “less than 98 mEq/l” is unsuitable, “98 to 110 mEq/l” is the normal range, and “over 110 mEq/l” is unsuitable. Regarding inorganic phosphorous, “less than 2.5 mg/dl” is unsuitable, “2.5 to 4.5 mg/dl” is the normal range, and “over 4.5 mg/dl” is unsuitable. Regarding total homocysteine, “less than 3.7 nmo/ml” is unsuitable, “3.7 to 13.5 nmo/ml” is the normal range, and “over 13.5 nmo/ml” is unsuitable. Regarding RF quantification, “20 U/ml or less” is the normal range, and “over 20 U/ml” is unsuitable. Regarding ASO, “239 IU/ml or less” is the normal range, and “over 239 IU/ml” is unsuitable. Regarding hemoglobin, in the case of males, “less than 13.1 g/dl” is unsuitable, “13.1 to 17.9 g/dl” is the normal range, and “over 17.9 g/dl” is unsuitable, and in the case of females, “less than 12.1 g/dl” is unsuitable, “12.1 to 15.9 g/dl” is the normal range, and “over 15.9 g/dl” is unsuitable. Regarding the hematocrit, in the case of males, “less than 38.5%” is unsuitable, “38.5 to 52.8%” is the normal range, and “over 52.8%” is unsuitable, and in the case of females, “less than 35.5%” is unsuitable, “35.5 to 46.9%” is the normal range, and “over 46.9%” is unsuitable. Regarding CEA, “5.0 ng/ml or less” is the normal range, and “over 5.0 ng/ml” is unsuitable. Regarding α-FP, “0 to 10 ng/ml” is the normal range, and “over 10 ng/ml” is unsuitable. Regarding CA19-9, “37 U/ml or less” is the normal range, and “over 37 U/ml” is unsuitable. Regarding CA125 (female), “35 U/ml or less” is the normal range, and “over 35 U/ml” is unsuitable. Regarding CA15-3 (female), “30 U/ml or less” is the normal range, and “over 30 U/ml” is unsuitable. Regarding PSA, “0 to 4.0 ng/ml” is the normal range, and “over 4.0 ng/ml” is unsuitable. Regarding gastrin-releasing peptide precursor, “less than 46.0 pg/ml” is the normal range, and “46.0 pg/ml or greater” is unsuitable. Regarding cytokeratin 19 fragments, “3.5 ng/ml or less” is the normal range, and “over 3.5 ng/ml” is unsuitable. Regarding SCC, “2.0 ng/ml or less” is the normal range, and “over 2.0 ng/ml” is unsuitable. Regarding NSE, “10.0 ng/ml or less” is the normal range, and “over 10.0 ng/ml” is unsuitable. Regarding STN, “45.0 U/m or less” is the normal range, and “over 45.0 U/m” is unsuitable. Regarding insulin, “less than 2.7 μU/ml” is unsuitable, “2.7 to 10.4 μU/ml or less” is the normal range, and “over 10.4 μU/ml” is unsuitable. These are merely examples, and lifestyle factor information optimum values are retained for other similar health states.

### <Hardware Architecture>

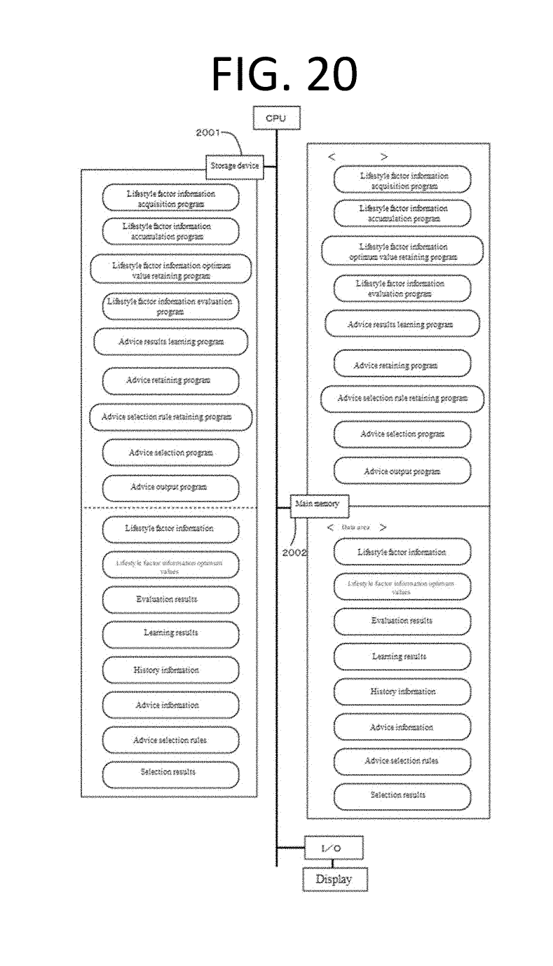

As shown in FIG. 20, a lifestyle factor information acquisition program, a lifestyle factor information accumulation program, a lifestyle factor information optimum value retention program, a lifestyle factor information evaluation program, an advice results learning program, an advice retention program, an advice selection rule retention program, an advice selection program, and an advice output program are stored in a storage device ((2001), a hard disk or ROM device for example), and furthermore, as data, the lifestyle factor information items, lifestyle factor information optimum values, evaluation results, learning results, history information, advice information, advice selection rules, and advice selection results are also retained in the storage device. Furthermore, when the present device is started, each of the abovementioned programs and necessary data are loaded from the storage device to the main memory (2002), and the present device can be operated as a computer. A new advice recording program for recording new advice may also be stored.

### <Processing Flow>

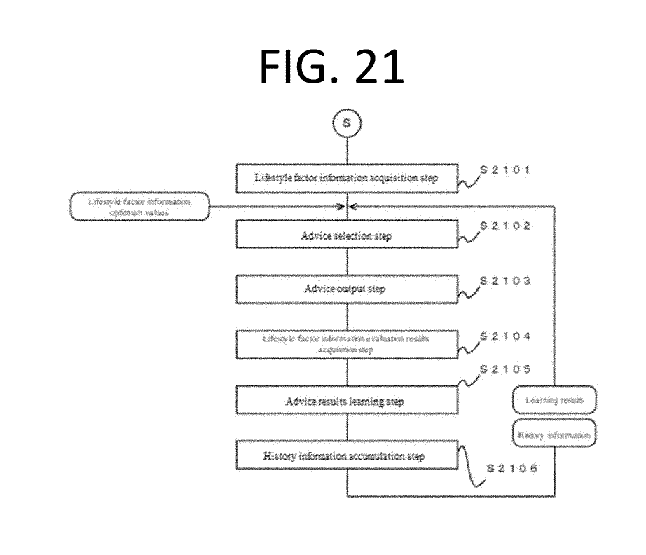

FIG. 21 is a flow chart showing the processing flow of the present embodiment. The processing flow of the present embodiment includes a lifestyle factor information acquisition step (S2101), an advice selection step (S2102), an advice output step (S2103), a lifestyle factor information evaluation results acquisition step (S2104), an advice results learning step (S2105), and a history information accumulation step (S2106).

The lifestyle factor information acquisition step is a step in which lifestyle factor information, which is information affecting the physiological activity of a user and/or information indicating the degree of health of the user, is acquired from the user.

The advice selection step is a step in which advice is selected using retained advice selection rules with the history information of the lifestyle factor information and the lifestyle factor information optimum values retained for those lifestyle factors being used as variables.

The advice output step is a step in which advice selected in the advice selection step is output.

The lifestyle factor information evaluation results acquisition step is a step in which the lifestyle factor information is evaluated using lifestyle factor information optimum values, and evaluation results are obtained.

The advice results learning step is a step in which the relationship between the evaluation results and the advice information corresponding to the evaluated lifestyle factor information is learned.

The history information accumulation step is a step in which the acquired lifestyle factor information is associated with advice information, which corresponds to the acquisition period of the lifestyle factor information and has been output, and the associated lifestyle factor information is accumulated as history information.

After the history information accumulation step, the processing returns once again to the advice selection step based on the learning results and history information, and these steps are repeated.

### <Summary>

Through the above configuration, advice results can be learned through artificial intelligence (AI), the precision of advice selection can be increased, and updates can be made.

1. A cancer prevention/improvement advice device comprising:
a lifestyle factor information acquisition unit for acquiring, from a user, lifestyle factor information, which is information affecting the physiological activity of the user and/or information indicating a degree of health of the user;
a lifestyle factor information accumulation unit for accumulating the acquired lifestyle factor information as history information;
a lifestyle factor information optimum value retaining unit for retaining lifestyle factor information optimum values, which are optimum values for each lifestyle factor directed at the purpose of cancer prevention and improvement;
an advice retaining unit for retaining a plurality of advice items for more closely approaching the optimum value of each lifestyle factor information;
an advice selection rule retaining unit for retaining advice selection rules, which are rules for selecting the retained advice using the history information of the lifestyle factor information and the lifestyle factor information optimum values retained for the lifestyle factors as variables;
an advice selection unit for selecting advice using the retained advice selection rules with the history information of the lifestyle factor information and the lifestyle factor information optimum values retained for the lifestyle factors being used as variables; and
an advice output unit for outputting advice selected by the selection unit.

2. A cancer prevention/improvement advice device comprising:
a lifestyle factor information acquisition unit for acquiring, from a user, lifestyle factor information, which is information affecting the physiological activity of the user and/or information indicating the degree of health of the user;
a lifestyle factor information accumulation unit for associating the acquired lifestyle factor information with a below-described advice information that is output corresponding to the acquisition period of the lifestyle factor information, and accumulating the associated lifestyle factor information as history information;
a lifestyle factor information evaluation unit for evaluating the lifestyle factor information using a below-described lifestyle factor information optimum value, and obtaining evaluation results;
an advice results learning unit for learning a relationship between the evaluation results and the advice information corresponding to the evaluated lifestyle factor information;
a lifestyle factor information optimum value retaining unit for retaining lifestyle factor information optimum values, which are optimum values for each lifestyle factor directed at the purpose of cancer prevention and improvement;
an advice retaining unit for retaining a plurality of advice items for more closely approaching the optimum value of each lifestyle factor information;
an advice selection rule retaining unit for retaining advice selection rules, which are rules for selecting the retained advice using the history information of the lifestyle factor information, the lifestyle factor information optimum values retained for the lifestyle factors, and the learning results;
an advice selection unit for selecting advice using the retained advice selection rules with the history information of the lifestyle factor information and the lifestyle factor information optimum values retained for the lifestyle factors being used as variables; and
an advice output unit for outputting advice selected by the selection unit.

3. The cancer prevention/improvement advice device according to claim 2, further comprising a new advice recording unit for recording new advice in the advice retaining unit.

4. The cancer prevention/improvement advice device according to claim 1, wherein the lifestyle factor information acquisition unit acquires, as lifestyle factor information, any one or more of information including body measurement results (BMI, abdominal girth, visceral fat, body fat percentage), body status examination results (blood pressure, visual acuity, intraocular pressure, pulmonary function, hearing capability, hair conditions), urinalysis results, stool examination results, number of times of stool passage, stool color, stool characteristics, blood biochemical test results, serological examination results, tumor marker examination results, diabetes mellitus examination results, cytological examination results, fundus camera examination results, breast examination results, ultrasound examination results, gastrointestinal tract examination results, chest X-ray examination results, CT examination results, MRI examination results, PET examination results, rectal examination results, rectal temperature, electrocardiogram examination results, equilibrium function examination results, arteriosclerosis evaluation results, osteoporosis examination results, female hormone examination results, risk marker examination results, and genetic information.

5. The cancer prevention/improvement advice device according to claim 1, wherein the lifestyle factor information acquisition unit acquires, as lifestyle factor information, any one or more of information including the light quality and brightness of the living environment, amount of noise in the living environment, room temperature of the living environment, humidity of the living environment, light quality and brightness of the workplace environment, amount of noise in the workplace environment, room temperature of the workplace environment, humidity of the workplace environment, meal content, calorie intake from meals, food product ingredients and amount ingested with meals, number of meals, timing of meals, details of ingested supplements, intake calories from ingested supplements, ingested supplement ingredients and amounts from ingested supplements, number of times of ingestion of ingested supplements, ingestion timing of ingested supplements, quality of sleep, amount of sleep, type of exercise and amount of exercise, quality of work and amount of work, working hours, amount of movement at work, commute quality and amount, evaluation results from company at work (salary increase, job promotion, job transfer, change in assignment), presence or lack of unemployment and career change, quality and amount of training to maintain and increase physical strength, quality and amount of walking around town, quality and amount of shopping, quality and amount of contact with the natural environment, quality and amount of entertainment, quality and amount of friendships, quality and amount of marital relations, type and amount of accidents, presence or lack of residence changes, presence or lack of marriage and divorce, purchase history of expensive articles and expensive goods, quality and amount of learning for obtaining qualifications, presence or lack of qualification acquisition, and change in relationship with customers for cases of company management and self-employment.

6. The cancer prevention/improvement advice device according to claim 1, wherein the lifestyle factor information acquisition unit acquires, as lifestyle factor information, any one or more of information including types and amounts of injuries and illnesses, types and amounts of drugs that are being taken in accordance with injuries and illnesses, frequency and time length of hospital visits according to injuries and illnesses, types and amounts of diagnoses performed in accordance with injuries and illnesses, types and amounts of surgeries performed in accordance with injuries and illnesses, devices carried on the body in accordance with injuries and illnesses, assistance tools, type and amount of bandages and the like.

7. The cancer prevention/improvement advice device according to claim 1, wherein the lifestyle factor information acquisition unit acquires, as lifestyle factor information, any one or more of information including voice quality of a user, voice magnitude of the user, an amount of speech by the user, status of autonomic nerves, mood of the user, level of satisfaction with sex life, embraced image of an item, mood when getting out of bed, mood when falling asleep, mood during sleep, mood during work, mood on holidays, mood during meals, mood when defecating, effective body temperature, coldness of hands and feet, and ease of respiration.

8. The cancer prevention/improvement advice device according to claim 1, wherein the lifestyle factor information acquisition unit comprises a meal information acquisition means for acquiring meal information, which is a type of lifestyle factor information, from a photograph of a meal.

9. The cancer prevention/improvement advice device according to claim 1, wherein the lifestyle factor information acquisition unit comprises an exercise data acquisition means for acquiring exercise data, which is a type of lifestyle factor information, from an exercise amount measuring instrument such as a pedometer.

10. The cancer prevention/improvement advice device according to claim 1, wherein the lifestyle factor information acquisition unit comprises a sleep data acquisition means for acquiring sleep data, which is a type of lifestyle factor information, from a sleep measuring instrument.

11. The cancer prevention/improvement advice device according to claim 1, wherein the lifestyle factor information acquisition unit comprises a stress data acquisition means for acquiring stress data, which is a type of lifestyle factor information, from a stress measuring instrument.

12. The cancer prevention/improvement advice device according to claim 1, wherein the lifestyle factor information acquisition unit comprises a biological data acquisition means for acquiring biological data, which is a type of lifestyle factor information, from a body composition meter.

13. (canceled)

14. The cancer prevention/improvement advice device according to claim 1, wherein for a case in which the lifestyle factor information is acquired as a numeric value, the lifestyle factor information accumulation unit comprises a first statistical processing means for determining, on the basis of past numeric values, an average value, a dispersion, a deviation value of data each time, a t-distribution, an F-distribution, and a chi-squared distribution.

15. The cancer prevention/improvement advice device according to claim 14, further comprising, for a case in which the lifestyle factor information is acquired as a numeric value, a lifestyle factor information cross-correlation coefficient acquisition unit for acquiring a lifestyle factor information cross-correlation coefficient, which is a cross-correlation coefficient for the lifestyle factor information accumulated by the lifestyle factor information accumulation unit.

16. The cancer prevention/improvement advice device according to claim 14, wherein the advice selection rule retaining unit has an advice first selection rule changing means for changing the retained advice selection rule on the basis of an average value, a dispersion, a deviation value of data each time, a t-distribution, an F-distribution, and a chi-squared distribution determined by the first statistical processing means of the lifestyle factor information accumulation unit.

17. The cancer prevention/improvement advice device according to claim 14, wherein the lifestyle factor information accumulation unit further comprises:
an advice associated lifestyle factor information accumulation means for accumulating lifestyle factor information that is associated with advice output from the advice output unit and acquired; and
an advice lifestyle factor information correlation coefficient acquisition unit for acquiring an advice lifestyle factor information correlation coefficient, which is a correlation coefficient between the associated advice and the lifestyle factor information.

18. The cancer prevention/improvement device according to claim 17, wherein the advice selection rule retaining unit comprises a second advice selection rule changing means for changing the retained advice selection rule on the basis of the advice lifestyle factor information correlation coefficient acquired by the advice lifestyle factor information correlation coefficient acquisition unit.

19. A cancer prevention/improvement advice system comprising:
the cancer prevention/improvement advice device according to claim 1, further comprising a history information output unit for outputting, through a network, the history information of the lifestyle factor information accumulated by the lifestyle factor information accumulation unit;
a history information acquisition unit for acquiring, through a network, the history information output from the history information output unit; and
a central server having:
a central server correlation coefficient calculation unit for statistically processing the acquired history information, and calculating a lifestyle factor information cross-correlation coefficient; and
a central server correlation coefficient output unit for outputting the calculated correlation coefficient.

20. The cancer prevention/improvement advice system according to claim 19, wherein the central server comprises a second statistical processing unit for determining, on the basis of the acquired history information, an average value of each lifestyle factor information, a dispersion, a deviation value of data each time, a t-distribution, an F-distribution, and a chi-squared distribution.

21. The cancer prevention/improvement advice system according to claim 19, wherein the central server further comprises:
an advice selection command acquisition unit for acquiring an advice selection rule change command, which is a command for changing the advice selection rule retained by the advice selection rule retaining unit for each cancer prevention/improvement advice device on the basis of the correlation coefficient calculated by the central server correlation coefficient calculation unit; and
an advice selection rule change command output unit for outputting the acquired advice selection rule change command to each cancer prevention/improvement advice device;
and each cancer prevention/improvement advice device further comprises:
an advice selection rule change command acquisition unit for acquiring an advice selection rule change command output from an advice selection rule change command output unit; and
an advice selection rule change unit for changing, on the basis of an acquired advice selection rule change command, an advice selection rule retained by the advice selection rule retaining unit.
